(19) United States
(12) Patent Application Publication
(10) Pub. No.: US 2021/0398631 A1
(43) Pub. Date: Dec. 23, 2021

(54) SYSTEMS AND METHODS FOR DISPLAYING A PATIENT SPECIFIC REPORT

(71) Applicant: GENOMIND, INC., King of Prussia, PA (US)

(72) Inventors: David S. KRAUSE, King of Prussia, PA (US); Daniel DOWD, King of Prussia, PA (US)

(21) Appl. No.: 17/349,797

(22) Filed: Jun. 16, 2021

Related U.S. Application Data
(60) Provisional application No. 63/040,665, filed on Jun. 18, 2020.

(51) Int. Cl. G16H 15/00; G06T 11/20; G06F 3/0481; G16H 10/60; G16H 20/70

(52) U.S. Cl. CPC G16H 15/00; G06T 11/206; G06T 2200/24; G16H 10/60; G16H 20/70; G06F 3/04817

(57) ABSTRACT

Systems and method for displaying a patient specific report are provided. The report includes a determination of an allele status for genomic loci. Each respective loci associates with an efficacy of a treatment for at least one neuropsychiatric disorder and one or more treatments of the at least one disorder. A respective graphical chart displays for each corresponding disorder including a respective first axis, a respective second axis, and respective data plots. The respective first axis segments into bins. Each respective bin associates with a respective independent first subset of loci and represents a respective treatment class. The second respective axis is associated with a characteristic of an efficacy of a treatment for the corresponding disorder. Each respective data plot represents a corresponding treatment, is within the corresponding bin, and independently located on the first respective axis based on an allele status for the respective independent first subset of loci.

### CROSS-REFERENCE TO RELATED APPLICATION

The present application claims priority to U.S. Provisional Patent Application No. 63/040,665, entitled “Systems and Methods for Displaying a Patient Specific Report,” filed Jun. 18, 2020, which is hereby incorporated by reference in its entirety.

### TECHNICAL FIELD

The present disclosure relates to the field of pharmacogenetics and, more specifically, to methods and systems for performing multiplexed single nucleotide polymorphism (SNP) detection assays.

### BACKGROUND

Not all patients react to a therapy in a uniform and beneficial manner. A number of factors including age, gender, ethnicity, and environmental and/or behavioral factors can influence the therapeutic efficacy and adverse reactions of therapeutic agents. Importantly, genetic variations among patients have been shown to account for variable drug reactions. Meyer, Urs A., “Pharmacogenetics and adverse drug reactions,” The Lancet 356:1667-71 (2000). For example, citalopram is one of the most commonly prescribed drugs for treating mental illness, such as depression. However, in populations with certain permutations of the gene CYP2C19 (2-4% Caucasians and 8-13% of Asians), administering a normal dosage of citalopram poses a significant risk of drug overexposure and adverse reaction. As such, a dosage adjustment is necessary for these patients. Thus, it is oftentimes beneficial for clinicians to have patients' pertinent genetic profiles available when making decisions, such as prescribing and dosing therapeutic agents.

Over the past thirty years, precision medicine has grown substantially, facilitated particularly by advances in molecular genetics and genotyping technologies. Modern genotyping technology allows for rapid detection and measurement of genetic variations, such as single nucleotide polymorphisms (SNPs), across a large span of the human genome. Over one-hundred million SNPs have been identified in the human population (Auton, A., et al., Nature, 526:68-74 (2015)), making them the most common type of genetic variation in humans. SNPs occur normally throughout the human genome and are mostly clinically insignificant. However, a relatively small portion of SNPs have been identified as important biomarkers associated with susceptibility to certain diseases and/or metabolism of different drugs. Syvanen, A., Nature Genetics, 37:S5-10 (2005). The SNP-based genotyping technology has been reportedly used in a variety of areas such as molecular diagnosis, prenatal analysis, predictive genetic testing, and in particular, pharmacotherapy, giving rise to the concept of “pharmacogenetics.” Roses, A., Nature, 405(3788):857-65 (2000).

Compared to conventional pharmaceutical approaches, where all patients diagnosed with a particular condition are prescribed a common therapy, pharmacogenetics provides personalized treatment based on the genotype profile specific to an individual patient. This allows for more accurate predictions about the patient's susceptibility of developing disease, the progression of the disease, and the patient's likely reaction to treatment. Accordingly, this approach helps clinicians achieve higher drug efficacy, increased drug tolerability, and reduced adverse reactions through better selection of therapeutic agents with dosages optimized for the individual patient.

Because of the rapidly growing understanding of key genetic biomarkers, like SNPs, and the impact the biology underlying the biomarkers has on drug metabolism, many pharmaceuticals have FDA-approved labels that list pertinent genetic biomarkers, warnings particular to specific patient populations, and information about metabolism of the drug relative to such genetic biomarkers and warnings. FDA-approved labels commonly also contain information about pharmacokinetic and pharmacodynamics drug interactions. FDA, “Table of Pharmacogenomic Biomarkers in Drug labeling.” For example, the FDA-approved label for aripiprazole, an atypical antipsychotic drug used to treat schizophrenia and other mental disorders, states that “[d]osage adjustments are recommended in patients who are known CYP2D6 poor metabolizers and in patients taking concomitant CYP3A4 inhibitors or CYP2D6 inhibitors or strong CYP3A4 inducers.” ABILIFY® Prescribing information, Otsuka America Pharmaceutical Inc., 03US19IBR0002, (2019). These genes encode important enzymes that metabolize pharmaceuticals in the liver. As such, best medical practices warrant clinicians to consult with drug labels constantly for gene-drug association information and comply with the label's instructions.

The difficulties associated with in scaling up genotyping assays pose a substantial hurdle to clinicians prescribing therapies with adverse gene-drug interactions, as they often consider multiple pharmaceutical solutions for a single patient. Each of these therapeutic options may be associated with multiple pharmacogenetic interactions, further compounding the problem. For example, when treating patients with similar symptoms of serious mental illness (SMI), clinicians often need to choose an appropriate prescription from a list of more than 30 FDA-approved drugs, many of which have known pharmacogenetic interactions. FDA, “Table of Pharmacogenomic Biomarkers in Drug labeling.” As such, only having a small set of these genes tested can hardly provide clinicians comprehensive and informative evaluation of the patient's drug profile.

For instance, mental illnesses are highly prevalent in the United States, and a major public health concern impacting nearly one in five adults. Serious mental illness (SMI), defined as a mental, behavioral, or emotional disorder resulting in serious functional impairment that substantially interferes with or limits one or more major life activities, is also prevalent in the U.S. More than ten million adults, representing 4.2% of the adult U.S. population, have been diagnosed with SMI. NIH, Mental Illness, November 2017. SMI costs more than $193 billion per year in lost earnings in the U.S. Major depressive disorder (MDD), bipolar disorder, schizophrenia, schizoaffective disorder, and other SMIs are associated with increased mortality from various causes, including but not limited to suicide. John A. et al., Schizophr Res., 199:154-62 (2018); Laursen T M et al., J Clin Psychiatry, 68(6):899-907 (2007). In military veterans, posttraumatic stress disorder and traumatic brain injury also increase the risk of suicidal behavior. Wilks C R et al., J Psychiatr Res., 109:139-44 (2019). Because the distinction between serious and any mental illness is not always apparent, even mental illnesses which are not typically thought of as serious may be associated with excess mortality. For example, attention-deficit hyperactivity disorder (ADHD) is associated with excess mortality. In part this may be due to co-morbidities, but this excess remains even when accounting for co-morbid mental health diagnoses. The excess mortality in ADHD is driven mostly by unnatural causes, including accidents. Dalsgaard S. et al., Lancet, 385(9983):2190-96 (2015).

Many individuals suffering with SMI do not respond adequately or completely to initial therapy. For example, among patients with MDD, response to initial treatment fails to occur in approximately half of all individuals; remission is even less frequent. Trivedi M H et al., Am J Psych, 163:28-40 (2006). In MDD, work-related disability and productivity loss are critical determinants of patient quality of life, and contribute significantly to the human and economic costs caused by this disease. Lee et al., J Affect Disord, 227:406-15 (2018). Schizophrenia, another SMI, follows a fairly consistent natural history characterized by initial response to antipsychotic drugs, but subsequent non-adherence, deterioration and recurrent episodes of psychosis. Lieberman J A., J Clin Psychiatry, 67(10):e14 (2006).

In patients with SMI, pharmacogenetic testing has the potential to assist in the selection of drugs which are more likely well tolerated, and to avoid serious adverse events (SAEs), as genetic variation is an important factor that influences the efficacy and tolerability (e.g., a ratio of benefit to risk profile) of pharmaceutical agents, including psychotropic drugs. For example, cytochrome p450 (CYP450) enzymes account for the metabolism of most pharmaceuticals. The identification and validation of these pharmacokinetic genes and of pharmacodynamic gene variants has enabled the emergence of precision medicine in psychiatry. Many pharmaceuticals, including many psychotropic drugs, now have biomarker warnings or precautions in their prescribing information, or contain pertinent information on the agent's metabolism, with respect to the effect of variants of genes encoding for CYP450 enzymes on the drug's exposure. Some product labels also contain information regarding the drug's ability to influence the exposure of concomitantly administered drugs via inhibition or induction of CYP450 enzymes. As the Agency notes, “Pharmacogenomics can play an important role in identifying responders and non-responders to medications, avoiding adverse events, and optimizing drug dose.” FDA, Table of Pharmacogenomic Biomarkers in Drug Labeling.

For example, aripiprazole, a second generation (or atypical) antipsychotic drug, is indicated to treat schizophrenia, mania associated with bipolar disorder, and several other serious disorders. Aripiprazole's label states: “Dosage adjustments are recommended in patients who are known CYP2D6 poor metabolizers and in patients taking concomitant CYP3A4 inhibitors or CYP2D6 inhibitors or strong CYP3A4 inducers.” Aripiprazole's label recommends half the usual starting dose in CYP2D6 poor metabolizers, and the dosage may vary by a factor of eight in the presence of concomitant inducers or inhibitors of CYP450 enzymes. Abilify® Prescribing Information, 2018. Otsuka America Pharmaceutical Inc.

Inefficacy is an obvious potential consequence of underexposure. Alternatively, excessive exposure may be associated with common and manageable or infrequent and serious tolerability issues, such as orthostasis or tardive dyskinesia. One of the most common drugs used in psychiatry is citalopram. In CYP2C19 poor metabolizers (2-4% of Caucasians and 8-13% of Asians) the AUC exposure to citalopram may be doubled, increasing the risk of QT prolongation. CELEXA® (citalopram HBr) Prescribing Information, Forest Laboratories, Inc.

Other biomarkers may also have an important role in the safe use of psychotropics, including the avoidance of SAEs. One example is the presence of the HLA-B*1502 gene variant, which is associated with increased risk for severe and sometimes fatal skin reactions, such as Stevens-Johnson syndrome and toxic epidermal necrolysis, to carbamazepine and oxcarbazepine. Phillips E J et al., Clinical Pharmacogenetics Implementation Consortium Guideline for HLA Genotype and Use of Carbamazepine and Oxcarbazepine: 2017 Update, Clinical Pharmacology & Therapeutics (2018). Carbamazepine is indicated for epilepsy and trigeminal neuralgia, but is widely utilized as a mood stabilizer in bipolar disorder, and as adjunctive therapy in MDD. APA, Practice Guideline for the Treatment of Major Depressive Disorder (3rd ed. 2010). Carbamazepine extended release capsules have the additional indication of acute mania or mixed episodes associated with bipolar I disorder. This risk is highlighted in carbamazepine's current drug label, which contains a boxed warning, and specifically calls for biomarker screening in individuals of Asian descent.

When treating patients with mental illnesses and disorders, it would be beneficial for clinicians to have a patient-specific report that can provide personalized treatment recommendations based on interpretive analysis of a patient's genotype. Although various research and clinical studies have looked for diagnostic and therapeutic indicators in an almost overwhelming variety of genomic markers, gene expression markers and protein markers, this vast and growing body of data has proven difficult to interpret. Synthesizing the tremendous amount of information on possible risk factors and indicators to apply this information clinically to diagnose and/or treat patients is challenging. As such, there is a need for methods and systems for providing patient-specific reports enabling medical professionals to apply the most relevant medical therapy in a meaningful manner to their patients.

### SUMMARY

Given the above background, what is needed in the art are systems and methods for providing medical professionals patient specific reports for personalized treatment of neuropsychiatric disorders. The patient specific reports of the present disclosure provide for a method of reporting patient specific information including pharmacogenetic test results obtained for the patient, risk assessments for the patient's current medications and/or planned medications, dosage guidelines, and alternative medicines to the patient's current or planned medications in a concise and comprehensive format.

The present disclosure provides improved systems and methods for displaying patient specific reports. Subjects are provided patient specific reports for one or more neuropsychiatric disorders. An allele detection system generates the patient specific reports based on determinations of allele statuses from genotyping assays for each subject. Such patient specific reports provide diagnosis summaries for the neuropsychiatric disorders in the form of graphical charts. Each graphical chart provides a comparison of pharmaceutical compositions based on a variety of pharmacogenetic parameters. The pharmaceutical compositions are positioned relatively, allowing for magnitudes of risks and/or benefits associated with a specific pharmaceutical composition to be readily determined according to its relative positioning. Reasoning for the relative positioning of each pharmaceutical composition is provided through a number of graphical icons that are selectively associated with each pharmaceutical composition. This is advantageous because complexities surrounding treatments of a neuropsychiatric disorder can be evaluated for multiple pharmaceutical compositions in a concise graphical chart, allowing medical professionals to digest complicated diagnosis without difficulty or need for reference materials.

In the present disclosure, a patient specific report for neuropsychiatric disorders is received from an allele detection system. In some embodiments, the allele detection system generates the patient specific report in response to performing a genotyping assay on a biological sample obtained from a subject, and further determining an allele status for a plurality of genomic loci for the subject. As such, the patient specific report includes the determination of the allele status and treatments for the neuropsychiatric disorders. The patient specific report is displayed on a device as a corresponding graphical chart for each of the neuropsychiatric disorders. Non-limiting examples of such graphical charts include an attention deficit hyperactivity disorder (ADHD) graphical chart, an anxiety disorder graphical chart, a bipolar disorder graphical chart, a depressive disorder, and a pain management graphical chart. Each respective graphical chart includes a first respective axis, a second respective axis, and one or more respective data plots that each represent a corresponding treatment. The one or more respective data plots populate an area of the respective graphical chart formed by the first respective axis and the second respective axis. The first respective axis is segmented into a respective plurality of bins, with each respective bin being associated with a respective independent subset of genomic loci and further representing a respective treatment class (e.g., therapy). The respective second axis is associated with a parameter or characteristic of an efficacy of a treatment for the neuropsychiatric disorder, such as a benefit-to-risk profile for the treatment. Each respective data plot is independently located relative to a position on the second respective axis based on the determined allele status. In this way, each respective bin associates information for a corresponding therapy, and each respective data plot provides a corresponding treatment for the corresponding therapy. For instance, as a first non-limiting example of such bins and data plots, a first bin represents a selective serotonin reuptake inhibitor therapy and a first data plot represents a Fluoxetine pharmaceutical composition, which is a treatment for the selective serotonin reuptake inhibitor therapy. As a second non-limiting example, a second bin represents a tricyclic antidepressant therapy and a second data plot represents Amitriptyline, which is a treatment for the tricyclic antidepressant therapy. This is highly advantageous since complex diagnosis information for treatments of a neuropsychiatric disorder is communicated through the relative position of the one or more respective data plots and thus is designed to allow for intuitive comparisons of the treatments without a need for detailed explanations or supplemental reference materials.

Accordingly, turning to more specific aspects of the present disclosure, systems and methods for displaying a patient specific report for neuropsychiatric disorders that concisely describes a diagnosis summary for a subject are provided. In some embodiments, the systems and methods described herein include receiving the patient specific report for the subject from an allele detection system. The patient specific report includes a determination of an allele status for a plurality of genomic loci, with each genomic loci being associated with a therapeutic efficacy of a treatment for a neuropsychiatric disorder and treatment of the neuropsychiatric disorder. On a device, the patient specific report is displayed as a respective graphical chart for each neuropsychiatric disorder of the patient specific report. Each respective graphical chart includes a respective first axis, a second respective axis, and one or more respective data plots. The respective first axis is segmented into a plurality of bins, each of which represents a respective treatment class and is associated with a respective subset of genomic loci. The respective second axis is associated with a parameter or characteristic of an efficacy of a treatment. Each respective data plot represents a corresponding treatment and is within the corresponding bin associated with the corresponding treatment. Thus, each respective graphical chart provides a visualization of treatments for the corresponding neuropsychiatric disorder.

In more detail, one aspect of the present disclosure provides a method of displaying a patient specific report for one or more neuropsychiatric disorders at a client device. The client device includes a display, one or more processors, and memory coupled to the one or more processors. The memory includes one or more programs configured to be executed by the one or more processors. The method includes receiving the patient specific report from an allele detection system. The patient specific report includes a determination of an allele status for a plurality of genomic loci for a subject. Each respective genomic loci in the plurality of genomic loci is associated with a therapeutic efficacy of a treatment, in a plurality of treatments, for at least one neuropsychiatric disorder in the one or more neuropsychiatric disorders. Each genomic loci is further associated with one or more treatments of the at least one neuropsychiatric disorder. A graphical user interface is displayed on the display of the client device. The graphical user interface includes a respective graphical chart for each corresponding neuropsychiatric disorder in the one or more neuropsychiatric disorders. Each respective graphical chart includes a respective first axis that is segmented into a respective plurality of bins. Each bin in the respective plurality of bins is associated with a respective independent first subset of genomic loci in the plurality of genomic loci. Each bin also represents a respective treatment class in a plurality of treatment classes. A respective second axis of the respective graphical chart is associated with a parameter or a characteristic of an efficacy of a treatment for the corresponding neuropsychiatric disorder associated with the respective graphical chart. The respective graphical chart further includes one or more respective data plots. Each respective data plot of the one or more respective data plots represents a corresponding treatment, in the plurality of treatments, is within the corresponding bin that is associated with the corresponding treatment, and is independently located relative to a position on the second axis based on an allele status determined for the subject, by the receiving, for the respective independent first subset of genomic loci associated with the corresponding bin, thereby visualizing one or more treatments of the one or more neuropsychiatric disorders.

In some embodiments, the respective graphical chart includes a frame of reference associated with a first therapeutic efficacy of the second axis. In some embodiments, the frame of reference includes a demarcation perpendicular to the second axis.

In some embodiments, the first therapeutic efficacy is an equilibrium efficacy.

In some embodiments, each respective genomic loci in a respective first subset of genomic loci of a bin of the respective graphical chart is associated with at least one respective data plot of the one or more respective data plots.

In some embodiments, each respective data plot that is within a respective first bin in the respective plurality of bins forms a corresponding cluster of data plots within the bin. The respective first bin is associated with a unique location on the respective first axis.

In some embodiments, the respective second axis includes a first boundary associated with a first magnitude of a largest detrimental change in efficacy and a second magnitude of a second boundary associated with a largest beneficial change in efficacy. In some embodiments, the first magnitude and the second magnitude are equal. In some embodiments, the respective second axis of each respective graphical chart is the same second axis.

In some embodiments, a distance from the frame of reference to the location of a respective data plot corresponds to a deviation in efficacy of the corresponding treatment relative to the first efficacy of the frame of reference.

In some embodiments, the distance from the frame of reference is either in a first direction indicating a positive deviation in efficacy or a second direction indicating a negative deviation in efficacy.

In some embodiments, a relation between the distance from the frame of reference and the deviation in efficacy is either a linear relation or a logarithmic relation.

In some embodiments, each respective data plot of the one or more respective data plots consumes a corresponding region of the graphical chart.

In some embodiments, the first therapeutic efficacy of the respective second axis is associated with a first hue. A relative distance from the frame of reference to each respective data plot shifts a color of the respective data plot from the first hue to a second hue as a function of the relative distance.

In some embodiments, an area of the consumed region for each respective data plot is the same area. In some embodiments, an area of the consumed region for each respective data plot is associated with a variability in the efficacy.

In some embodiments, each respective treatment in the one or more treatments is a corresponding pharmaceutical composition. The respective region of a respective data plot includes a text portion providing an identification of the pharmaceutical composition of the corresponding treatment of the respective data plot.

In some embodiments, the identification includes a class of the pharmaceutical composition.

In some embodiments, each respective treatment in the one or more treatments is a corresponding pharmaceutical composition. For a subset of data plots of the one or more respective data plots, each respective region of a corresponding data plot in the subset of data plots includes one or more graphical icons in a plurality of graphical icons. Furthermore, each respective graphical icon in the plurality of graphical icons represents a parameter or characteristic of the corresponding treatment represented by the corresponding data plot.

In some embodiments, the subset of data plots includes each data plot of the one or more respective data plots that satisfies a threshold parameter or characteristic of a respective efficacy.

In some embodiments, the threshold parameter or characteristic includes a first threshold magnitude as a function of the first magnitude, a second threshold magnitude as a function of the second magnitude, a threshold parameter or characteristic associated with the subject, or a threshold parameter or characteristic associated with a respective pharmaceutical composition.

In some embodiments, the plurality of graphical icons includes a first graphical icon representing a change in weight of the subject. In some embodiments, the first graphical icon is associated with a second generation antipsychotic pharmaceutical composition.

In some embodiments, the plurality of graphical icons includes a second graphical icon representing a deviation in efficacy based on a characteristic of the subject. In some embodiments, the second graphical icon is associated with a brain-derived neurotrophic factor antidepressant pharmaceutical composition.

In some embodiments, the plurality of graphical icons incudes a third graphical icon representing a positive deviation in efficacy of a pharmaceutical composition. In some embodiments, the plurality of graphical icons includes a fourth graphical icon representing a negative deviation in efficacy of a pharmaceutical composition. In some embodiments, the third and fourth graphical icons are each associated with an antidepressant pharmaceutical composition, an attention deficit hyperactivity disorder pharmaceutical composition, or an antipsychotic pharmaceutical composition. In some embodiments, the third and fourth graphical icons are each associated with an antidepressant pharmaceutical composition or an antipsychotic pharmaceutical composition.

In some embodiments, the plurality of graphical icons includes a fifth graphical icon representing a positive deviation in tolerance with consumption of a pharmaceutical composition. In some embodiments, the plurality of graphical icons includes a sixth graphical icon representing a negative deviation in tolerance with consumption of a pharmaceutical composition. In some embodiments, the fifth and sixth graphical icons are each associated with an opioid pharmaceutical composition.

In some embodiments, the plurality of graphical icons includes a seventh graphical icon representing an adverse side effect with consumption of a pharmaceutical composition.

In some embodiments, the plurality of graphical icons includes an eighth graphical icon representing a warning associated with consumption of a pharmaceutical composition. In some embodiments, the eighth graphical icon is associated with an anticonvulsant pharmaceutical composition.

In some embodiments, the plurality of graphical icons includes a ninth graphical icon representing a positive deviation in exposure with consumption of a pharmaceutical composition. In some embodiments, the plurality of graphical icons includes a tenth graphical icon representing a negative deviation in exposure with consumption of a pharmaceutical composition.

In some embodiments, the plurality of graphical icons includes an eleventh graphical icon representing a negative deviation in exposure with consumption of a cytochrome P450 1A2 inhibitor pharmaceutical composition.

In some embodiments, the plurality of graphical icons includes a twelfth graphical icon representing a prodrug pharmaceutical composition.

In some embodiments, the respective graphical chart includes a figure legend illustrating a subset of the plurality of graphical icons. In some embodiments, the figure legend is displayed beneath the respective first axis and the respective second axis. In some embodiments, the subset of graphical icons of the figure legend contains each graphical icon in the plurality of graphical icons.

In some embodiments, each respective graphical icon in the plurality of graphical icons is provided a corresponding rank. Furthermore, each respective graphical icon of a respective region of a corresponding data plot is displayed in an ordered form by rank.

In some embodiments, the displaying the respective graphical chart for each neuropsychiatric disorder includes displaying a plurality of respective graphical charts of the one or more treatments.

In some embodiments, a data plot representing a corresponding treatment is unassociated with a bin in the respective plurality of bins.

In some embodiments, the one or more neuropsychiatric disorders of the patient specific report includes a depressive disorder, a bipolar disorder, an anxiety disorder, a post-traumatic stress disorder, a schizophrenia disorder, an attention deficit hyperactivity disorder, an autism spectrum disorder, a treatment resistant form of one of the aforementioned disorders, or a combination thereof.

In some embodiments, a first bin in the respective plurality of bins represents selective serotonin reuptake inhibitors. In some embodiments, a second bin in the respective plurality of bins represents serotonin-norepinephrine reuptake inhibitors. Furthermore, in some embodiments, a third bin in the respective plurality of bins represents tricyclic antidepressants.

In some embodiments, a bin in the respective plurality of bins represents selective serotonin reuptake inhibitors, serotonin-norepinephrine reuptake inhibitors, tricyclic antidepressants, or monoamine oxidase inhibitors.

In some embodiments, a first bin in the respective plurality of bins represents selective serotonin reuptake inhibitors. Each respective treatment in the one or more treatments is a corresponding pharmaceutical composition. The first bin includes a data plot for Citalopram, Escitalopram, Fluoxetine, Paroxetine, or Sertraline.

In some embodiments, a first bin in the respective plurality of bins represents selective serotonin reuptake inhibitors. Each respective treatment in the one or more treatments is a corresponding pharmaceutical composition. The first bin includes a respective data plot for Citalopram, Escitalopram, Fluoxetine, Paroxetine, and Sertraline.

In some embodiments, a first bin in the respective plurality of bins represents serotonin-norepinephrine reuptake inhibitors. Each respective treatment in the one or more treatments is a corresponding pharmaceutical composition. The first bin includes a data plot for Desvenlafaxine, Duloxetine, Levomilnacipran, or Venlafaxine.

In some embodiments, a first bin in the respective plurality of bins represents serotonin-norepinephrine reuptake inhibitors. Each respective treatment in the one or more treatments is a corresponding pharmaceutical composition. The first bin includes a respective data plot for Desvenlafaxine, Duloxetine, Levomilnacipran, and Venlafaxine.

In some embodiments, a first bin in the respective plurality of bins represents tricyclic antidepressants. Each respective treatment in the one or more treatments is a corresponding pharmaceutical composition. The first bin includes a data plot for Amitriptyline, Amoxapine, Desipramine, Doxepin, Imipramine, Nortriptyline, Protriptyline, or Trimipramine.

In some embodiments, a first bin in the respective plurality of bins represents tricyclic antidepressants. Each respective treatment in the one or more treatments is a corresponding pharmaceutical composition. The first bin includes a respective data plot for Amitriptyline, Amoxapine, Desipramine, Doxepin, Imipramine, Nortriptyline, Protriptyline, and Trimipramine.

In some embodiments, the plurality of genomic loci includes a locus from one or more human genes selected from the group consisting of CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, and MC4R. In some embodiments, the plurality of genomic loci includes at least one locus from each of the following human genes: CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, and MC4R.

In some embodiments, the plurality of genomic loci includes at least one locus corresponding to a SNP selected from the group consisting of rs7997012, rs6265, rs4680, rs2470890, rs2069514, rs35694136, rs2069526, rs762551, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs1135840, rs16947, rs1135824, rs35742686, rs3892097, rs5030655, rs5030867, rs5030865, rs5030656, rs1065852, rs5030863, rs5030862, rs5030865(T), rs774671100, rs28371706, rs61736512, rs1058164, rs59421388, rs28371725, rs35599367, rs776746, rs10264272, rs41303343, rs1061235, rs2395148, rs489693, rs1801131, rs1801133, rs25531, rs63749047, and rs1902023. In some embodiments, the plurality of genomic loci includes the loci corresponding to SNPs rs7997012, rs6265, rs4680, rs2470890, rs2069514, rs35694136, rs2069526, rs762551, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs1135840, rs16947, rs1135824, rs35742686, rs3892097, rs5030655, rs5030867, rs5030865, rs5030656, rs1065852, rs5030863, rs5030862, rs5030865(T), rs774671100, rs28371706, rs61736512, rs1058164, rs59421388, rs28371725, rs35599367, rs776746, rs10264272, rs41303343, rs1061235, rs2395148, rs489693, rs1801131, rs1801133, rs25531, rs63749047, and rs1902023.

In some embodiments, the plurality of genomic loci includes a locus from one or more human genes selected from the group consisting of CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, MC4R, 5HT2C, ADRA2A, ANK3, CACNA1C, DRD2, GRIK1, OPRM1, UGT1A4, and ABCB1. In some embodiments, the plurality of genomic loci includes at least one locus from each of the following human genes: CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, MC4R, 5HT2C, ADRA2A, ANK3, CACNA1C, DRD2, GRIK1, OPRM1, UGT1A4, and ABCB1.

In some embodiments, the plurality of genomic loci includes at least one locus corresponding to a SNP selected from the group consisting of rs7997012, rs3813929, rs1045642, rs2032583, rs1800544, rs10994336, rs6265, rs1006737, rs4680, rs2470890, rs2069514, rs35694136, rs2069526, rs762551, rs12720461, rs2069526, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs7900194, rs16947, rs1135840, rs1135824, rs35742686, rs3892097, rs5030655, rs5030867, rs5030865, rs5030656, rs1065852, rs5030863, rs5030862, rs5030865, rs774671100, rs28371706, rs16947, rs61736512, rs1058164, rs16947, rs59421388, rs1135840, rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023. In some embodiments, the plurality of genomic loci includes the loci corresponding to SNPs rs7997012, rs3813929, rs1045642, rs2032583, rs1800544, rs10994336, rs6265, rs1006737, rs4680, rs2470890, rs2069514, rs35694136, rs2069526, rs762551, rs12720461, rs2069526, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs7900194, rs16947, rs1135840, rs1135824, rs35742686, rs3892097, rs5030655, rs5030867, rs5030865, rs5030656, rs1065852, rs5030863, rs5030862, rs5030865, rs774671100, rs28371706, rs16947, rs61736512, rs1058164, rs16947, rs59421388, rs1135840, rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

Another aspect of the present disclosure provides a system for displaying a patient specific report for one or more neuropsychiatric disorders. The system includes one or more processors, a display, and memory. The one or more programs are stored in the memory and configured to be executed by the one or more processors. The one or more programs including instructions for receiving the patient specific report from an allele detection system. The patient specific report includes a determination of an allele status for a plurality of genomic loci for a subject. Each respective genomic loci in the plurality of genomic loci is associated with a therapeutic efficacy of a treatment, in a plurality of treatments, for at least one neuropsychiatric disorder in the one or more neuropsychiatric disorders. Each genomic loci is further associated with one or more treatments of the at least one neuropsychiatric disorder. The one or more programs further include instructions for displaying a graphical user interface on the display of the client device. The graphical user interface includes a respective graphical chart for each corresponding neuropsychiatric disorder in the one or more neuropsychiatric disorders. Each respective graphical chart includes a respective first axis that is segmented into a respective plurality of bins. Each bin in the respective plurality of bins is associated with a respective independent first subset of genomic loci in the plurality of genomic loci. Each bin also represents a respective treatment class in a plurality of treatment classes. A respective second axis of the respective graphical chart is associated with a parameter or a characteristic of an efficacy of a treatment for the corresponding neuropsychiatric disorder associated with the respective graphical chart. The respective graphical chart further includes one or more respective data plots. Each respective data plot of the one or more respective data plots represents a corresponding treatment, in the plurality of treatments, is within the corresponding bin that is associated with the corresponding treatment, and is independently located relative to a position on the second axis based on an allele status determined for the subject, by the receiving, for the respective independent first subset of genomic loci associated with the corresponding bin. As such, the system visualizing one or more treatments of the one or more neuropsychiatric disorders.

Yet another aspect of the present disclosure provides a non-transitory computer readable storage medium. The non-transitory computer readable storage medium stores instructions, which when executed by a computer system, cause the computer system to perform a method. The method includes receiving the patient specific report from an allele detection system. The patient specific report includes a determination of an allele status for a plurality of genomic loci for a subject. Each respective genomic loci in the plurality of genomic loci is associated with a therapeutic efficacy of a treatment, in a plurality of treatments, for at least one neuropsychiatric disorder in the one or more neuropsychiatric disorders. Each genomic loci is further associated with one or more treatments of the at least one neuropsychiatric disorder. A graphical user interface is displayed on the display of the client device. The graphical user interface includes a respective graphical chart for each corresponding neuropsychiatric disorder in the one or more neuropsychiatric disorders. Each respective graphical chart includes a respective first axis that is segmented into a respective plurality of bins. Each bin in the respective plurality of bins is associated with a respective independent first subset of genomic loci in the plurality of genomic loci. Each bin also represents a respective treatment class in a plurality of treatment classes. A respective second axis of the respective graphical chart is associated with a parameter or a characteristic of an efficacy of a treatment for the corresponding neuropsychiatric disorder associated with the respective graphical chart. The respective graphical chart further includes one or more respective data plots. Each respective data plot of the one or more respective data plots represents a corresponding treatment, in the plurality of treatments, is within the corresponding bin that is associated with the corresponding treatment, and is independently located relative to a position on the second axis based on an allele status determined for the subject, by the receiving, for the respective independent first subset of genomic loci associated with the corresponding bin, thereby visualizing one or more treatments of the one or more neuropsychiatric disorders.

Another aspect of the present disclosure provides a method of displaying a patient specific report for a neuropsychiatric disorder at a client device. The client device includes a display, one or more processors, and memory coupled to the one or more processors. The memory includes one or more programs configured to be executed by the one or more processors. The method includes receiving the patient specific report from an allele detection system. The patient specific report includes a determination of an allele status for a plurality of genomic loci for a subject. Each respective genomic loci in the plurality of genomic loci is associated with a therapeutic efficacy of a treatment, in a plurality of treatments, for at least one neuropsychiatric disorder in the one or more neuropsychiatric disorders. Each genomic loci is further associated with one or more treatments of the at least one neuropsychiatric disorder. A graphical user interface is displayed on the display of the client device. The graphical user interface includes a respective graphical chart for each corresponding neuropsychiatric disorder in the one or more neuropsychiatric disorders. Each respective graphical chart includes a respective first axis that is segmented into a respective plurality of bins. Each bin in the respective plurality of bins is associated with a respective independent first subset of genomic loci in the plurality of genomic loci. Each bin also represents a respective treatment class in a plurality of treatment classes. A respective second axis of the respective graphical chart is associated with a parameter or a characteristic of an efficacy of a treatment for the corresponding neuropsychiatric disorder associated with the respective graphical chart. The respective graphical chart further includes one or more respective data plots. Each respective data plot of the one or more respective data plots represents a corresponding treatment, in the plurality of treatments, is within the corresponding bin that is associated with the corresponding treatment, and is independently located relative to a position on the second axis based on an allele status determined for the subject, by the receiving, for the respective independent first subset of genomic loci associated with the corresponding bin, thereby visualizing one or more treatments of the one or more neuropsychiatric disorders.

A further aspect of the present disclosure provides a method for treating a subject for a neuropsychiatric disorder. The method includes determining a mental health pharmacogenetic profile for the subject. The mental health pharmacogenetic profile is determined by obtaining or having obtained a biological sample from the subject. The method includes performing or having performed a genotyping assay on the biological sample to determine the allele status for a plurality of genomic loci for the subject. Each respective genomic loci in the plurality of genomic loci is associated with a therapeutic efficacy of a treatment, in a plurality of treatments, for a neuropsychiatric disorder. Furthermore, the method includes receiving, at a client device including a display, one or more processors, and memory coupled to the one or more processors, a patient specific report. The patient specific report includes the determination of the allele status for the plurality of genomic loci and one or more treatments for the neuropsychiatric disorder. The method includes displaying, on the display, in a graphical user interface, a respective graphical chart for each corresponding neuropsychiatric disorder in the one or more neuropsychiatric disorders. Each respective graphical chart includes a respective first axis, a respective second axis, and one or more respective data plots. The respective first axis is segmented into a respective plurality of bins. Each bin in the respective plurality of bins is associated with a respective independent first subset of genomic loci in the plurality of genomic loci. Each bin in the respective plurality of bins also represents a respective treatment class in a plurality of treatment classes. The respective second axis is associated with a parameter or a characteristic of an efficacy of a treatment for the corresponding neuropsychiatric disorder associated with the respective graphical chart. Furthermore, each respective data plot of the one or more respective data plots represents a corresponding treatment, in the plurality of treatments, is within the corresponding bin that is associated with the corresponding treatment, and is independently located relative to a position on the second axis based on an allele status determined for the subject, by the receiving, for the respective independent first subset of genomic loci associated with the corresponding bin. When a respective data plot indicates that a first treatment, in the plurality of treatments, is better suited for treatment of the neuropsychiatric disorder in the individual than a second treatment, in the plurality of treatments, administering the first treatment to the subject. When the data plot indicates that the second treatment is better suited for treatment of the neuropsychiatric disorder in the individual that the first treatment, administering the second treatment to the subject.

### BRIEF DESCRIPTION OF THE DRAWINGS

FIG. 1 illustrates a block diagram illustrating an embodiment of a system for displaying a specific patient report, in accordance with an embodiment of the present disclosure;

FIG. 2 illustrates an allele detection system for generating a patient specific report, in accordance with an embodiment of the present disclosure;

FIG. 3 illustrates a client device for displaying a patient specific report, in accordance with an embodiment of the present disclosure;

FIG. 4 provides a first flow chart of methods for displaying a specific patient report, in accordance with an embodiment of the present disclosure, in which optional elements are indicated by dashed boxes and/or lines;

FIG. 5 provides a second flow chart of methods for displaying a specific patient report, in accordance with an embodiment of the present disclosure, in which optional elements are indicated by dashed boxes and/or lines;

FIG. 6A illustrates a first user interface of a specific patient report, in accordance with an embodiment of the present disclosure; and

FIG. 6B illustrates a second user interface of a specific patient report, in accordance with an embodiment of the present disclosure.

Like reference numbers refer to corresponding parts throughout the several views of the drawings.

### DETAILED DESCRIPTION

In treating neuropsychiatric disorders, it is important for clinicians to have a patient specific report that can provide personalized treatment recommendations based on interpretive analysis of a respective patient's genotype. Although various research and clinical studies have looked for diagnostic and therapeutic indicators in an almost overwhelming variety of genomic markers, gene expression markers, and protein markers, this vast and growing body of data has proven difficult to interpret. Most physicians are unable to synthesize the tremendous amount of information on possible risk factors and indicators in order to apply this information clinically to diagnose and/or treat patients. As such, there is a need for patient-specific reports to enable a medical professional to apply the most relevant genetic, epigenetic, transcriptomic, proteomic and functional imaging tests in a meaningful manner to their patients.

The present disclosure provides systems and methods for providing a patient specific report for one or more neuropsychiatric disorders. The patent specific report includes recommendations for a treatment of a neuropsychiatric disorder in the one or more neuropsychiatric disorders. Moreover, the patent specific report is generated based on an allege status of one or more tested genomic loci. Advantageously, by providing a comprehensive mental health pharmacogenetic profile, through evaluation of the improved combinations of gene and loci provided herein, clinicians can more intelligently select personalized pharmacotherapy for neuropsychiatric disorders. For example, many pharmaceutical compositions approved for treating neuropsychiatric disorders have FDA-approved labels that include therapeutic guidance relating to pertinent genetic biomarkers. Information about the allele status of these genetic biomarkers in a patient leads to optimized pharmacotherapy with higher efficacy, lower tolerability and/or lesser adverse reactions to one or more pharmaceutical compositions. Unfortunately, a comprehensive source of this information is not readily available for clinicians, because conventional genotyping assays only test for a limited subset of the genes and alleles necessary to make informed choices when deciding between the many therapeutic options for treatment of neuropsychiatric disorders. The methods of the present disclosure facilitate collection and analysis of this comprehensive data, which improves clinical outcomes for the treatment of neuropsychiatric disorders. As such, the methods provided herein are capable of providing comprehensive evaluation of available pharmacotherapy options based on the specific genotype of a patient. In addition, the methods provided herein represent a streamlined, nimble process for generating a patient-specific report based on the genotyping assay results that can be readily useful for medical practitioners in a clinical setting.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first graphical chart could be termed a second graphical chart, and, similarly, a second graphical chart could be termed a first graphical chart, without departing from the scope of the present disclosure. The first graphical chart and the second graphical chart are both graphical charts, but they are not the same graphical chart.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which includes nucleic acids reflecting the genotype of the subject with respect to the loci described herein. Examples of biological samples include, but are not limited to, blood samples, saliva samples, buccal cell samples, and the like. A sample can be a liquid sample or a solid sample (e.g., a cell or tissue sample).

As used herein, the term “genomic locus” or “locus” refers to a position (e.g., a site) within a genome, i.e., on a particular chromosome. In some embodiments, a locus refers to a single nucleotide position within a genome, i.e., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, i.e., one copy on the maternal autosomal chromosome and one copy on the paternal autosomal chromosome.

As used herein, the term “allele” refers to a particular sequence of one or more nucleotides at a genomic locus. Because normal mammalian cells have diploid genomes, a normal mammalian genome (e.g., a human genome) will have two alleles for each genomic locus, which may be the same or different. When a mammal has the same allele at both copies of a locus, they are homozygous for the allele. When an organism has different alleles at their two copies of a locus, they are heterozygous for the two alleles. Accordingly, in some embodiments, the “allele status” or “allelic status” of a mammal at a genomic locus may be homozygous for an allele (e.g., the wild type or most prevalent allele) or heterozygous (e.g., having one copy of the wild type or most prevalent allele and one copy of a variant allele or less prevalent allele). In some embodiments, determining the allelic status of a locus of a mammal includes determining whether the mammal carries, e.g., has at least one copy of, an allele with a known pharmacogenetic effect. In some embodiments, when it is determined that the mammal carries at least one copy of the particular allele, it is determined whether the mammal is homozygous or heterozygous for the particular allele. For example, in some embodiments, this is done when the pharmacogenetic effect of the particular allele is known to be dosage-dependent, that is, when the pharmacogenetic effect of the allele is different when the subject is homozygous for the particular allele than when the subject is heterozygous for the allele. However, in other embodiments, determining the allelic status of a locus of a subject only includes determination of whether the subject carries the particular allele, regardless of the copy number of the particular allele.

As used herein, the term “equally spaced” means that a distance from a first feature to a corresponding second feature is the same for successive pairs of features unless expressly stated otherwise.

As used herein, the term “dynamically” means an ability to update a program while the program is currently running.

Additionally, the terms “client,” “patient,” “subject,” and “user” are used interchangeably herein unless expressly stated otherwise.

Moreover, the terms “patient specific report” and “graphical user interface” are used interchangeably herein unless expressly stated otherwise.

In addition, the terms “therapy” and “treatment” are used interchangeably herein unless expressly stated otherwise.

Furthermore, when a reference number is given an “i^{th}” denotation, the reference number refers to a generic component, set, or embodiment. For instance, a graphical icon termed “graphical icon i” refers to the i^{th} graphical icon in a plurality of graphical icons (e.g., a graphical icon 616-i in a plurality of graphical icons 616).

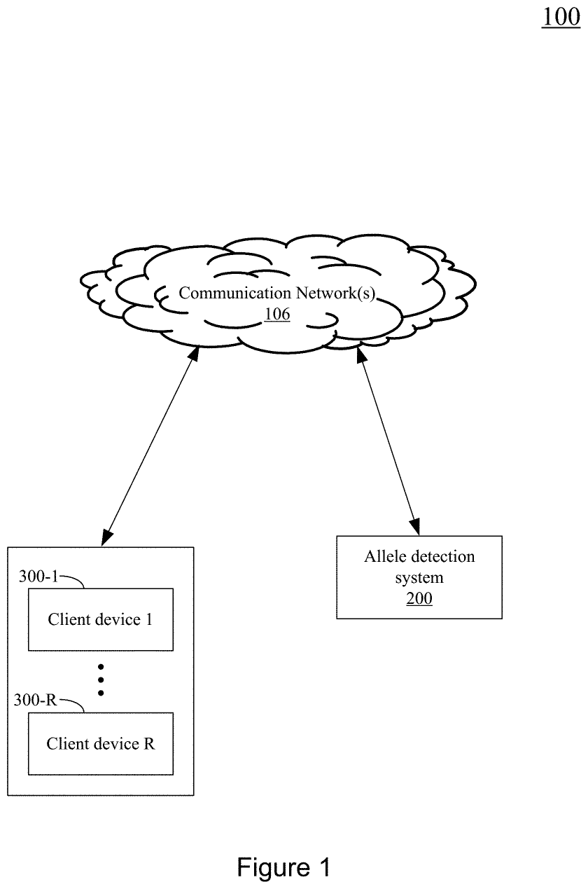

In the present disclosure, unless expressly stated otherwise, descriptions of devices and systems will include implementations of one or more computers. For instance, and for purposes of illustration in FIG. 1, a client device 300 is represented as single device that includes all the functionality of the client device 300. However, the present disclosure is not limited thereto. For instance, the functionality of the client device 300 may be spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the client device 300, and other devices and systems of the preset disclosure, and that all such topologies are within the scope of the present disclosure.

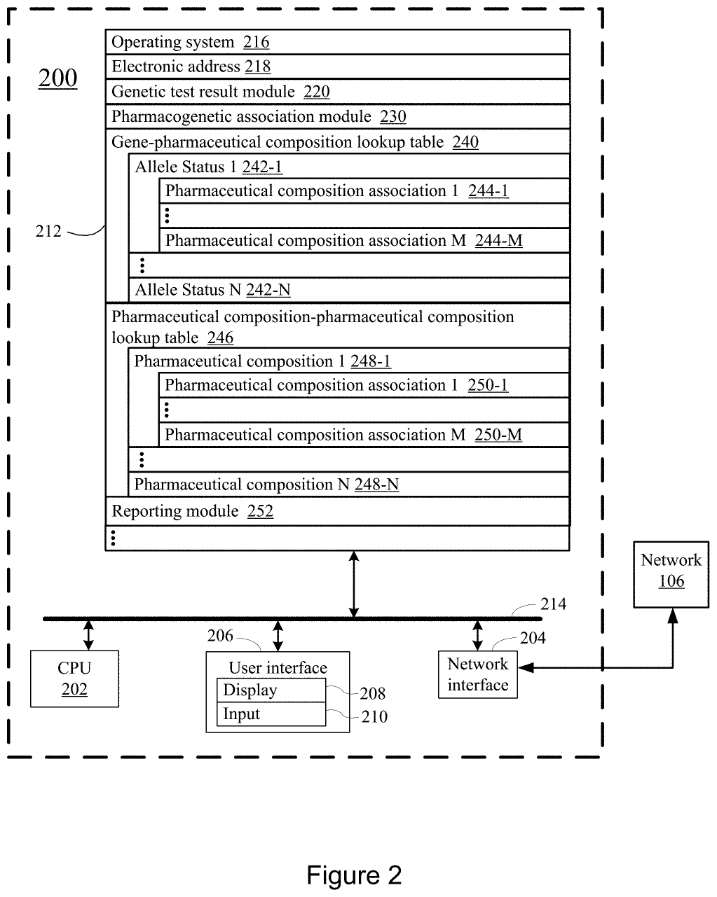

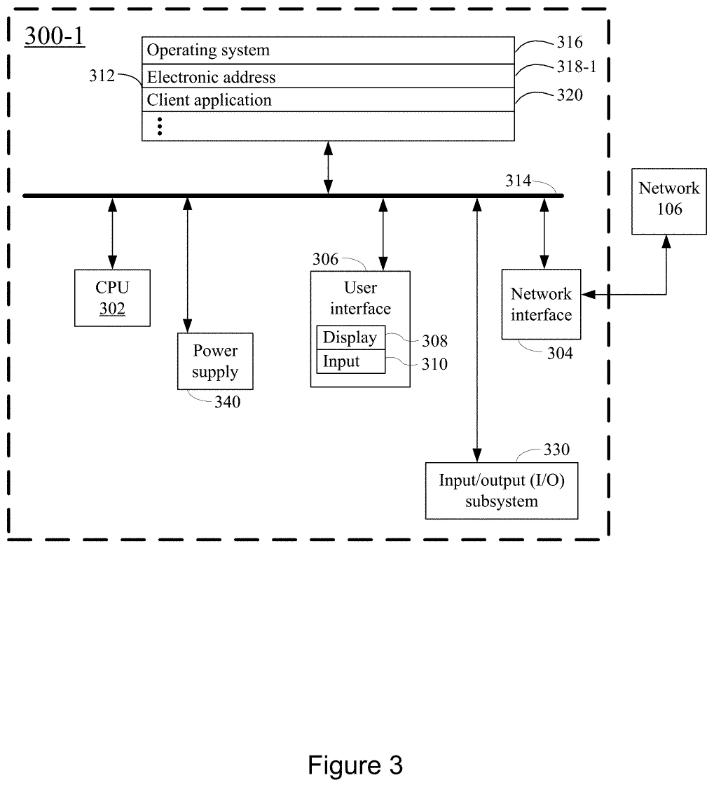

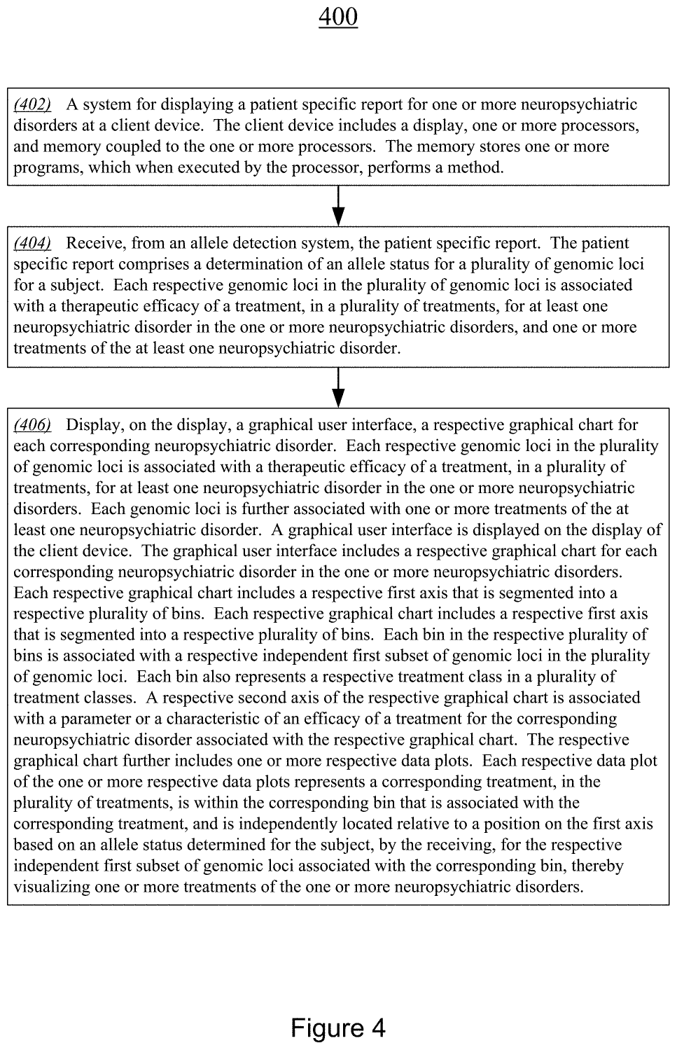

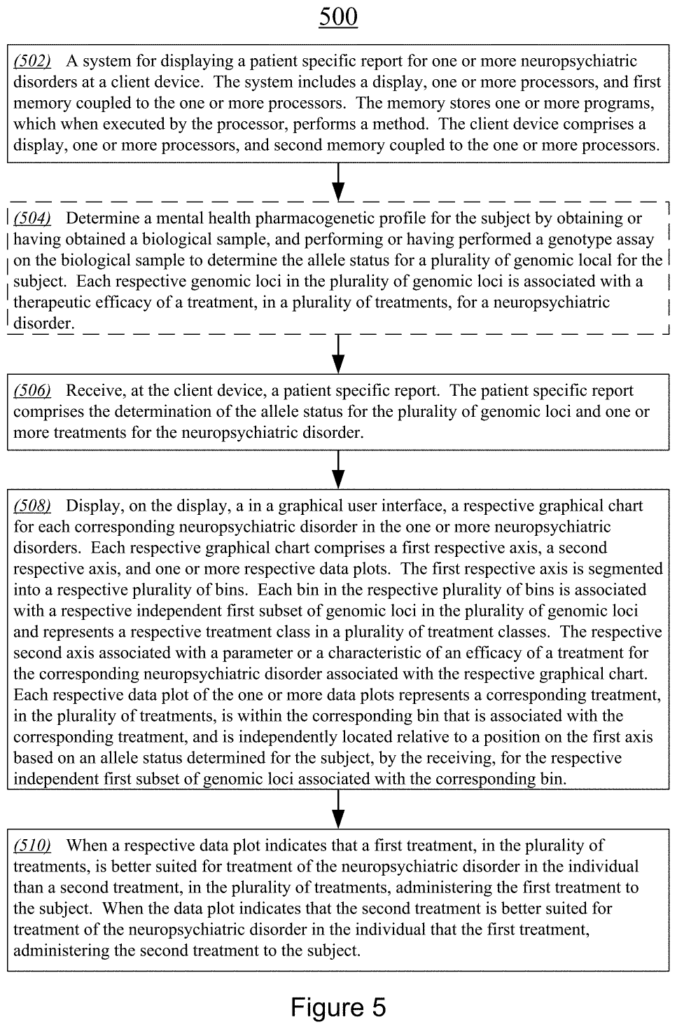

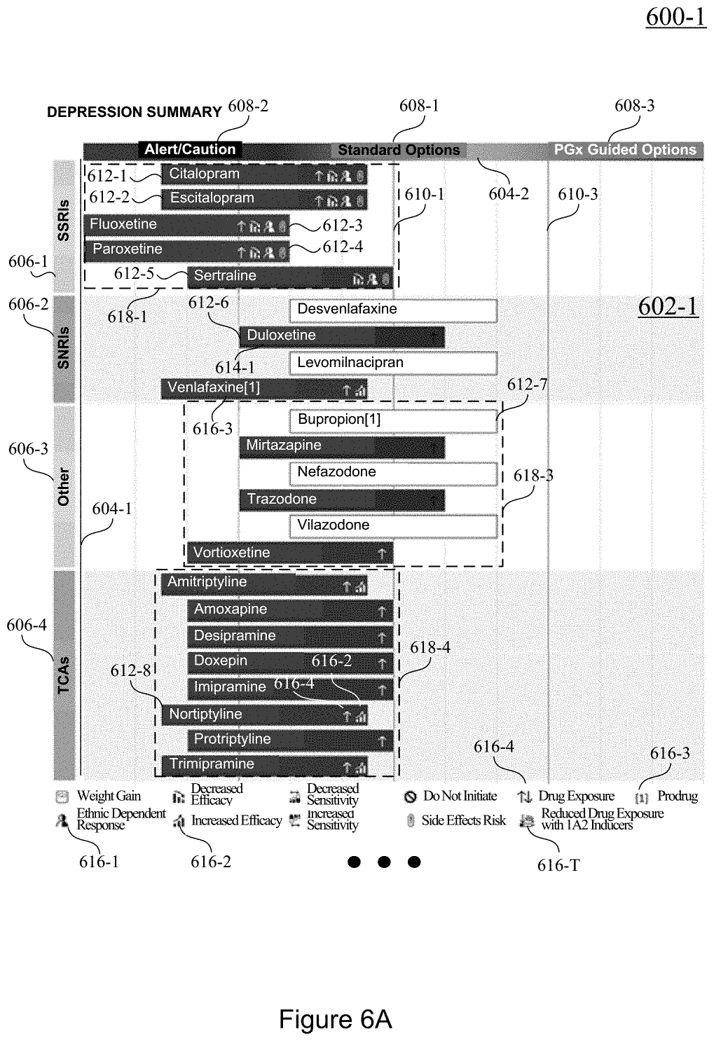

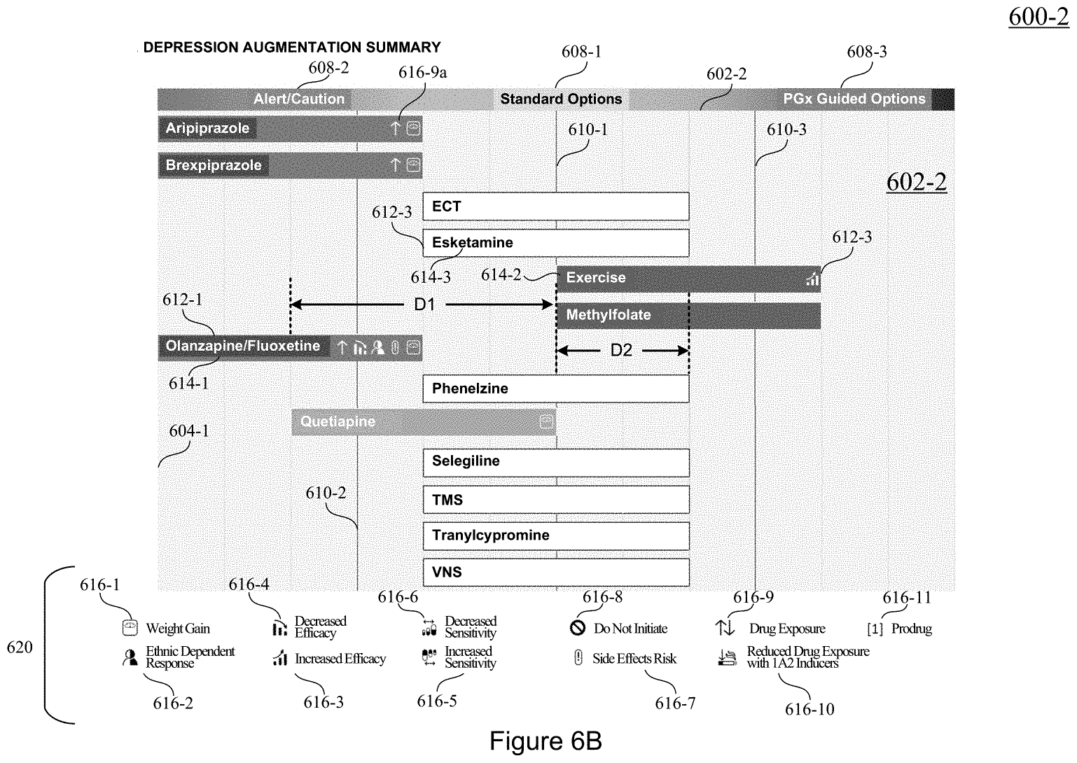

FIG. 1 depicts a block diagram of a distributed client-server system (e.g., distributed client-server system 100) according to some embodiments of the present disclosure. The system 100 facilitates providing a patient specific report (e.g., patient specific report 600 of method 400 of FIG. 4, patient specific report of method 500 of FIG. 5, patient specific report 600-1 of FIG. 6A, patient specific report 600-2 of FIG. 6B, etc.) to a subject (e.g., client device 300, client device 300-1 of FIG. 3, etc.). In some embodiments, the system 100 determines an allele status at a plurality of genomic loci in a genotyping reaction (e.g., a status of an allele 242 of FIG. 2 for a patient). Each respective genomic loci in the plurality of genomic loci is associated with a therapeutic efficacy of a treatment for at least one neuropsychiatric disorder in a plurality of neuropsychiatric disorders (e.g., pharmaceutical composition association 244 of FIG. 2). In response to the determined allele status, the system 100 provides the patient specific report (e.g., the first patient specific report 600-1 of FIG. 6A) to one or more client devices 300. The patient specific report 600 includes the determination of the allele status for the plurality of genomic loci for a subject. Furthermore, the patient specific report 600 includes one or more treatments of the at least one neuropsychiatric disorder. In this way, the patient specific report 600 provides simple, precise, and readily digestible information for medical practitioners in a clinical setting.

The system 100 facilitates providing a patient specific report 600 for a population of subjects (e.g., client devices 300). The patient specific report 600 is provided to a subject through a graphical user interface (GUI) (e.g., user interface 374 of FIG. 3) that is displayed through a display of a respective client device 300 (e.g., display 376 of FIG. 3). For each neuropsychiatric disorder of the at least one neuropsychiatric disorder of a respective patient specific report 600, a corresponding graphical chart (e.g., graphical chart 600-1 of FIG. 6A, graphical chart 600-2 of FIG. 6B, etc.) is displayed through the GUI.

Of course, other topologies of the system 100 are possible. For instance, in some embodiments, any of the illustrated devices and systems can in fact constitute several computer systems that are linked together in a network, or be a virtual machine or container in a cloud-computing environment. Moreover, rather than relying on a physical communications network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

Referring to FIG. 1, in some embodiments, a distributed client-server system 100 includes one or more client devices 300 (e.g., a first client device 300-1, a second client device 300-2, . . . , a R^{th} client device 300-R, etc.), hereinafter “client device,” each of which is associated with at least one corresponding subject (e.g., a patient of a corresponding patient specific report, a medical practitioner associated with a patient, etc.).

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

Now that a distributed client-server system 100 has generally been described, an exemplary allele detection system 200 for generating a specific patient report 600 will be described with reference to FIG. 2.

In various embodiments, the allele detection system 200 includes one or more processing units (CPUs) 202, a network or other communications interface 204, and memory 212.

Memory 212 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 212 may optionally include one or more storage devices remotely located from the CPU(s) 202. Memory 212, or alternatively the non-volatile memory device(s) within memory 212, includes a non-transitory computer readable storage medium. Access to memory 212 by other components of the allele detection system 200, such as the CPU(s) 202, is, optionally, controlled by a controller. In some embodiments, memory 212 can include mass storage that is remotely located with respect to the CPU(s) 202. In other words, some data stored in memory 212 may in fact be hosted on devices that are external to the allele detection system 200, but that can be electronically accessed by the allele detection system 200 over an Internet, intranet, or other form of network 106 or electronic cable using communication interface 204.

In some embodiments, the memory 212 of the allele detection system 200 for generating a patient specific report 600 stores:
an operating system 216 (e.g., ANDROID, iOS, DARWIN, RTXC, LINUX, UNIX, OS X, WINDOWS, or an embedded operating system such as VxWorks) that includes procedures for handling various basic system services;
an electronic address 218 associated with the allele detection system 200 that identifies the allele detection system 200;
a genetic test result module 220 for obtaining a set of genetic test results (e.g., a determination of an allele status), including one or more variant alleles (e.g., each status 242 of each variant alleles detectable in a genotypic assay) measured from a patient and specific to one or more neuropsychiatric disorders;
a pharmacogenetic association module 230 for associating the one or more variant alleles status 242 with one or more treatments of the one or more neuropsychiatric disorders (e.g., using a gene-pharmaceutical composition lookup table 240 with pharmaceutical composition associations 244 associated with each variant allele status 242), and using a pharmaceutical composition-pharmaceutical composition lookup table 246 with pharmaceutical composition associations 250 associated with each pharmaceutical composition 248; and
a reporting module 252 for generating a patient-specific report 600 including the one or more treatments of the one or more neuropsychiatric disorders.

An electronic address 218 is associated with the allele detection system 200, which is utilized to at least uniquely identify the allele detection system 200 from other devices and components of the distributed system 100. For instance, in some embodiments, the electronic address 218 is utilized to receive a request from a client device 300 to generate and/or communicate a patient specific report 600.

A genetic test result module 220 obtains a set of genetic test results including a determination of an allele status 242 for one or more alleles of the gene-pharmaceutical composition lookup table 240 detected in a genotypic assay measured from a patient and specific to one or more neuropsychiatric disorders. The genetic test result module 220 can provide the determination of an allele status 242 from a plurality of data (e.g., data derived from a biological sample from a patient), provided the determination in a pre-evaluated form, or both. In some embodiments, the determination of an allele status 242 is conducted at the allele detection system 200, or received from a remote device that is external to the allele detection system 200, such as a remote device associated with an allele detection facility. As a non-limiting example, in some embodiments, the genetic test result module provides a determination of an allele status 242 including a determination of a first allele status 242-1 and a third allele status 242-3 for the plurality of genomic loci of a patient. Accordingly, based on a determination of the allele status 242, a portion of the gene-pharmaceutical composition lookup table 240 associated with the first allele status 242-1 and the third allele status 242-3 is retrieved for use in generating a patient specific report 600 for the patient.

A pharmacogenetic association module 230 forms and/or receives one or more associations between an allele status 242 for a respective loci and a corresponding treatment of the one or more neuropsychiatric disorders. Given that, the determination of an allele status 242 can provide a deviation in efficacy of a pharmaceutical composition 248, such as a deviation in tolerance or a deviation in exposure, and in certain cases, differential therapeutic effects, including differences in risks of adverse side effects, the pharmacogenetic association module 230 stores and provides information related known pharmacogenetic association. For instance, in some embodiments, the pharmacogenetic association module 230 communicates with one or more databases associated with genomic loci and/or and stores information derived from one or more publications associated with genomic loci in order to provide an association between the determination of an allele status 242 for the patient and further associations between one or more allelic effects (e.g., pharmaceutical composition associations 244) and pharmaceutical compositions 248. In some embodiments, the pharmacogenetic association module 230 evaluates information received and/or derived from the one or more databases and/or the one or more publications associated with genomic loci in order to form an aggregation of pharmacogenetic associations.

In some embodiments, the one or more neuropsychiatric disorders include a depressive disorder (e.g., patient specific report 600-1 providing a “depression summary” report, patient specific report 600-2 providing an “depression augmentation summary” report, etc.), a bipolar disorder, an anxiety disorder, a post-traumatic stress disorder, a schizophrenia disorder, an attention deficit hyperactivity disorder (ADHD), an autism spectrum disorder, a treatment resistant form of one of the aforementioned disorders, or a combination thereof. Additional details and information related to one or more neuropsychiatric disorders of the present disclosure are described in more detail infra, with particular reference to block 404 of FIG. 4 and block 504 of FIG. 5.

Referring back go FIG. 2, a gene-pharmaceutical composition lookup table 240 includes a data structure that facilitators storing data pertaining to a plurality of allele statuses 242 of a plurality of genomic loci. Each respective allele status 242 is a variant of a corresponding genomic loci in the plurality of genomic loci. Accordingly, in some embodiments, the gene-pharmaceutical composition lookup table 240 includes each allele status 242 that is detectable from a genotyping assay, and therefore, included in a determination of an allele status 242 for a patient.

Each allele status 242 includes one or more pharmaceutical composition associations 244 that includes information related to an association between a determination of a respective allele status 242 and a corresponding pharmaceutical composition 248. In some embodiments, the pharmaceutical composition association 244 includes data related to a genomic loci of a respective allele status 242, one or more affected subgroups, one or more pharmaceutical compositions 248 associated with the respective allele status 242, a description of the pharmaceutical composition association 244, a description of information that supports therapeutic management recommendations, a description of information that indicates a potential impact on safety or response, a description of information that demonstrates a potential impact on pharmacokinetic properties, or a combination thereof. Furthermore, in some embodiments, a respective pharmaceutical composition association 244 is associated with a corresponding class of treatment (e.g., a class of pharmaceutical compositions) and/or a corresponding treatment (e.g., a pharmaceutical composition). In some embodiments, the description of the pharmaceutical composition associations 244 includes a text-based description, such as a description of adverse side effect risks. Additionally, in some embodiments, the pharmaceutical composition association 244 includes data related to one or more courses of therapy (e.g., a class of treatments, a treatment), one or more modifications to the one or more courses of therapy, or both. As a non-limiting example, in some embodiments, a first allele status 242-1 is associated with a determination at the CYP2D6 gene. This first allele status 242-1 includes a first pharmaceutical composition association 244-1 describing: an association with a corresponding treatment of a first pharmaceutical composition 248-1 of Venlafaxine; affected subgroups are poor metabolizers; a result alters systemic parent pharmaceutical composition and metabolite concentrations; and consideration of lowering a threshold dosage. The first allele status 242-1 further includes a second pharmaceutical composition association 244-2 describing: an association with a corresponding treatment of a second pharmaceutical composition 248-2 of Paroxetine; affected subgroups are either poor metabolizers, intermediate metabolizers, or ultra-rapid metabolizers; and a result in higher systemic concentrations.

A pharmaceutical composition-pharmaceutical composition lookup table 246 includes a data structure that facilitates storing a plurality of data pertaining to a plurality of pharmaceutical compositions 248. Each pharmaceutical composition 248 includes one or more pharmaceutical composition associations 250, which forms a linking association between the pharmaceutical composition 248 and one or more allele status 242.

In some embodiments, the pharmaceutical composition-pharmaceutical composition lookup table 246 further stores a plurality of data pertaining to a treatment for the one or more neuropsychiatric disorders, such as a treatment of exercise (e.g., data plot 612-2 of FIG. 6B). In this way, the one or more pharmaceutical composition associations 250 associated with the treatment form an association between the treatment and the one or more allele status 242.

In some embodiments, the pharmacogenetic association module 230, the gene-pharmaceutical composition lookup table 240, the pharmaceutical composition-pharmaceutical composition lookup table 246, or a combination thereof is dynamically updated. For instance, when a new pharmaceutical composition 248 is released, the pharmacogenetic association module 230, the gene-pharmaceutical composition lookup table 240, the pharmaceutical composition-pharmaceutical composition lookup table 246, or the combination thereof automatically updates the relative portions therein to reflect the recent release changes of the pharmaceutical composition 248 without requiring a forced update. Likewise, when a pharmaceutical composition association 244 is updated or released, the pharmacogenetic association module 230, the gene-pharmaceutical composition lookup table 240, the pharmaceutical composition-pharmaceutical composition lookup table 246, or the combination thereof automatically registers and implements the update without human intervention. A frequency and/or a triggering event at which the pharmacogenetic association module 230, the gene-pharmaceutical composition lookup table 240, the pharmaceutical composition-pharmaceutical composition lookup table 246, or the combination thereof is updated depends on a design of the present disclosure. For instance, in some embodiments, the frequency is a reoccurring basis, the triggering event is an actionable event at a client device 300 (e.g., in response to an instruction from a subject to update the pharmacogenetic association module 230, the gene-pharmaceutical composition lookup table 240, the pharmaceutical composition-pharmaceutical composition lookup table 246, or the combination thereof).

A reporting module 252 facilitates generating a patient specific report 600. In some embodiments, the reporting module 252 receives the determination of an allele status 242 from the genetic test result module 220, and generates the patient specific report 600 based on the received determination of the allele status 242. In some embodiments, the reporting module 252 considers a plurality of generation criteria when generating the patient specific report 600. In some embodiments, the generation criteria include a patient criteria (e.g., a patient criteria to exclude one or more treatments from the patient specific report 600 based on a known adverse reaction to the one or more treatments for the patient), a client device 300 criteria (e.g., a client device 300 criteria to limit a graphical chart to a predetermined number of treatments, receive the patient specific report 600 in a predetermined format, etc.), or both.

Each of the above identified modules and applications correspond to a set of executable instructions for performing one or more functions described above and the methods described in the present disclosure (e.g., the computer-implemented methods and other information processing methods described herein; method 400 of FIG. 4; method 500 of FIG. 5; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 212 optionally stores a subset of the modules and data structures identified above. Furthermore, in some embodiments, the memory 212 stores additional modules and data structures not described above.

It should be appreciated that the allele detection system 200 of FIG. 2 is only one example of an allele detection system 200, and that the allele detection system 200 optionally has more or fewer components than shown, optionally combines two or more components, or optionally has a different configuration or arrangement of the components. The various components shown in FIG. 2 are implemented in hardware, software, firmware, or a combination thereof, including one or more signal processing and/or application specific integrated circuits.

Referring to FIG. 3, an exemplary client device 300 is provided (e.g., first client device 300-1). A client device 300 includes one or more processing units (CPUs) 302, one or more network or other communication interfaces 304, memory 311 (e.g., random access memory and/or non-volatile memory) optionally accessed by one or more controllers, and one or more controllers, and one or more communication busses 314 interconnecting the aforementioned components.

In some embodiments, a client device 300 includes a mobile device, such as a mobile phone, a tablet, a laptop computer, a wearable device such as a smart watch, and the like. Alternatively, in some embodiments, the client device 300 is a desktop computer or other similar devices. Furthermore, in some embodiments, the client devices 300 (e.g. a first user device 300-1, a second user device 300-2, a third user device 300-3, etc.) communicate with a centralized client device 300 (e.g., a server client device 300) that facilitates allocating one or more patient specific reports 600 to one or more of the client devices 300. Moreover, in some embodiments, this centralized client device 300 receives and/or combines one or more patient specific reports 600 in order to communicate a single collective patient specific report 600. For instance, referring briefly to FIGS. 6A and 6B, in some embodiments, this centralized client device 300 combines the first patient specific report 600-1 of FIG. 6A and a second patient specific report 6B. Further, in some embodiments, each client device 300 enables a respective subject to provide information related to the respective subject (e.g., subject preferences, subject feedback, etc.).

In addition, the client device 300 includes a user interface 306. The user interface 306 typically includes a display device 308 for presenting media, such as a patient specific report 600, and receiving instructions from the subject operating the client device 300. In some embodiments, the display device 308 is optionally integrated within the client device 300 (e.g., housed in the same chassis as the CPU 302 and memory 312), such as a smart (e.g., smart phone) device. In some embodiments, the client device 300 includes one or more input device(s) 310, which allow the subject to interact with the client device 300. In some embodiments, input devices 310 include a keyboard, a mouse, and/or other input mechanisms. Alternatively, or in addition, in some embodiments, the display device 308 includes a touch-sensitive surface, e.g., where display 308 is a touch-sensitive display or client device 300 includes a touch pad.

In some embodiments, the client device 300 includes an input/output (I/O) subsystem 330 for interfacing with one or more peripheral devices with the client device 300. For instance, in some embodiments, audio is presented through an external device (e.g., speakers, headphones, etc.) that receives audio information from the client device 300 and/or a remote device (e.g., allele detection system 200), and presents audio data based on this audio information. In some embodiments, the input/output (I/O) subsystem 330 also includes, or interfaces with, an audio output device, such as speakers or an audio output for connecting with speakers, earphones, or headphones. In some embodiments, the input/output (I/O) subsystem 330 also includes voice recognition capabilities (e.g., to supplement or replace an input device 310).

In some embodiments, the client device 300 also includes one or more sensors (e.g., an accelerometer, a magnetometer, a proximity sensor, a gyroscope, etc.), an image capture device (e.g., a camera device or an image capture module and related components), a location module (e.g., a Global Positioning System (GPS) receiver or other navigation or geolocation system module/device and related components), or a combination thereof, and the like.

Memory 312 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 312 may optionally include one or more storage devices remotely located from the CPU(s) 302. Memory 312, or alternatively the non-volatile memory device(s) within memory 312, includes a non-transitory computer readable storage medium. Access to memory 312 by other components of the client device 300, such as the CPU(s) 302 and the I/O subsystem 330, is, optionally, controlled by a controller. In some embodiments, memory 312 can include mass storage that is remotely located with respect to the CPU 302. In other words, some data stored in memory 312 may in fact be hosted on devices that are external to the client device 300, but that can be electronically accessed by the client device 300 over an Internet, intranet, or other form of network 106 or electronic cable using communication interface 304.

In some embodiments, the memory 312 of the client device 300 stores:
an operating system 316 that includes procedures for handling various basic system services;
an electronic address 318 associated with the client device that identifies the client device; and
a client application 320 for generating content for display through a graphical user interface presented on the display 308 the client device 300.

An electronic address 318 is associated with the client device 300, which is utilized to at least uniquely identify the client device 300 from other devices and components of the distributed system 100. In some embodiments, the electronic address 318 associated with the client device 300 is used to determine a source of a communication received from (e.g., communicating feedback in response to a patient specific report 600) and/or provided to the client device 300 (e.g., receiving a patient specific report 600 from the allele detection system 200).

In some embodiments, a client application 320 is a group of instructions that, when executed by a processor (e.g., CPU(s) 302), generates content (e.g., a patient specific report 600) for presentation to the subject. In some embodiments, the client application 320 generates content in response to one or more inputs received from the subject through the user interface 306 of the client device 300. For instance, in some embodiments, the client application 320 includes a media presentation application for viewing the contents of a file or web application that includes the patient specific report 600.

Each of the above identified modules and applications correspond to a set of executable instructions for performing one or more functions described above and the methods described in the present disclosure (e.g., the computer-implemented methods and other information processing methods described herein; method 400 of FIG. 4; method 500 of FIG. 5; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 312 optionally stores a subset of the modules and data structures identified above. Furthermore, in some embodiments, the memory 312 stores additional modules and data structures not described above.

It should be appreciated that the client device 300 of FIG. 3 is only one example of a client device 300, and that the client device 300 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Now that a general topology of the distributed system 100 has been described in accordance with various embodiments of the present disclosures, details regarding some processes in accordance with FIGS. 4 and 5 will be described. FIG. 4 illustrates a first flow chart of methods (e.g., method 400) for generating and displaying a patient specific report 600 in accordance with embodiments of the present disclosure. Specifically, an exemplary method 400 for presenting genetic information that is a patient specific report 600 and relevant to treatment of one or more neuropsychiatric disorders is provided in accordance with some embodiments of the present disclosure. FIG. 5 illustrates a second flow chart of methods (e.g., method 500 of FIG. 5) for generating and displaying a patient specific report 600 in accordance with embodiments of the present disclosure. Specifically, an exemplary method 500 for a method for generating and displaying a patient specific report 600, and optionally, treating a subject (e.g., a patient of the patient specific report 600) for a neuropsychiatric disorder is provided in accordance with some embodiments of the present disclosure. In the flow charts, the preferred parts of the methods are shown in solid line boxes, whereas optional variants of the methods, or optional equipment used by the methods, are shown in dashed line boxes. As such, FIGS. 4 and 5 illustrate methods for generating and displaying a patient specific report 600.

Various modules in the memory 212 of the allele detection system 200, the memory 312 of a client device 300, or both perform certain processes of the methods described in FIGS. 4 and 5, unless expressly stated otherwise. Furthermore, it will be appreciated that the processes in FIGS. 4 and 5 can be encoded in a single module or any combination of modules.

Block 402. Referring to block 402 of FIG. 4, a system (e.g., distributed system 100 of FIG. 1) is provided for displaying a patient specific report (e.g., patient specific report 600-1 of FIG. 6A; patient 600-2 of FIG. 6B). The system 100 includes an allele detection system (e.g., allele detection system 200 of FIG. 1, allele detection system 200 of FIG. 2) and one or more client devices (e.g., client device 300 of FIG. 2, client device 300 of FIG. 3). The client device 300 includes a display (e.g., display 308 of FIG. 3), one or more processors (e.g., CPU(s) 302 of FIG. 3), and memory (e.g., memory 312 of FIG. 3) coupled to the one or more processors 302, such that a subject can view content through the display 308 of the client device 300. The memory 312 includes one or more programs configured to be executed by the one or more processors 302 (e.g., client application 320). When executed by the processor 302, the one or more programs (e.g., client application 320) performs a method (e.g., method 400 of FIG. 4, method 500 of FIG. 5, etc.). However, the present disclosure is not limited thereto.

For instance, in some embodiments, the present disclosure is conducted on the allele detection system 200 without a requirement for a client device 300. In this way, the allele detection system 200 subsumes a role (e.g., modules and components) of the client device 300 in conducting one or more processes (e.g., blocks) of the method 400, or a method 500 of FIG. 5, which would otherwise be conducted by the client device 300. Similarly, in some embodiments, the present disclosure is conducted on the client device 300 without a requirement for an allele detection system 200. In this way, the client device 300 subsumes a role of the allele detection system 200 in conducting one or more processes of the method 400, or the method 500 of FIG. 5, which would otherwise be conducted by the allele detection system 200.

Block 404. Referring to block 404, the method 400 includes receiving the patient specific report 600 from the allele detection system 200 (e.g., receiving the patient specific report 600 through communications network 106). In some embodiments, the client device 300 receives the patient specific report 600 in response to a request for the patient specific report 600 (e.g., a request to the allele detection system 200 and/or a database associated with the allele detection system 200). For instance, in some embodiments, the client device 300 communicates a request for a first patient specific report 600-1 in the form of an application programming interface (API) call to the allele detection system 200 or a database associated with the allele detection system 200. In response to the request, the allele detection system 200 generates the first patient specific report 600-1, or retrieves the first patient specific report 600-1, for communication to the client device 300, upon which the client device 300 receives the first patient specific report 600-1 for visualization on the display 308. However, the present disclosure is not limited thereto.

For instance, in some embodiments, the allele detection system 200 receives information related to a location of a subject associated with the client device 300 and/or the patient of the patient specific report 600, such as information obtained and/or derived from the electronic address 318 of the client device 300. In some embodiments, the allele detection system 200 obtains this information prior to generating the patient specific report 600. This allows the allele detection system 200 to communicate the patient specific report 600 in accordance with a determination that the generating of the patient specific report 600 has been conducted, therefore, the patient specific report 600 in condition for communication to the client device 300. In other words, in some embodiments, the allele detection system 200 can push the patient specific report 600 to the client device 300, the client device 300 can pull the patient specific report 600 from the allele detection system 200, or both. Moreover, the allele detection system 200 can generate the patient specific report 600 in response to a request for the patient specific report 600; the allele detection system 200 can generate, store, and then retrieve the patient specific report 600; or both.

The patient specific report 600 includes a determination of an allele status (e.g., a determination of one or more allele status 242 of FIG. 2) for a plurality of genomic loci for the patient. In some embodiments, the allele status 242 is determined from a biological sample obtained from the patient. In some embodiments, the biological sample includes, for example, buccal cells, saliva, blood, or a combination thereof. Exemplary systems and methods for obtaining the determination of the allele status 242 from the biological sample are described in detail in U.S. Provisional Patent Application No. 62/969,906, entitled “Methods and Systems for Multiplex Allele Detection,” filed Feb. 4, 2020, which is hereby incorporated by reference in its entirety.

As a non-limiting example, consider a plurality of allele statuses 242 of the gene-pharmaceutical composition lookup table 240 including a first allele status 242-1 including a first pharmaceutical composition association 244-1, a second allele status 242-2 including the first pharmaceutical composition association 244-1, a third allele status 242-3 including a second pharmaceutical composition association 244-2, and a fourth allele status 242-4 including a third pharmaceutical composition association 244-3 and a fourth pharmaceutical composition association 244-4. A first biological sample is obtained from a first patient, and a second biological sample is obtained from a second patient. In response, the genetic test result module 220 of allele detection system 200 evaluates the first biological sample and the second biological sample to determine an allele status 242 for each respective biological sample. A first determination of an allele status 242 of the first biological sample includes a determination of the first allele status 242-1 and the third allele status 242-2 for the first patient, whereas a second determination of an allele status 242 of the second biological sample includes a determination of the second allele status 242-2 and the third allele status 242. Accordingly, a unique determination of an allele status 242 is provided for each patient, such that the patient specific report 600 is tailored exclusively to a respective patient.

In some embodiments, the determination of the allele status 242 obtained is applied to a course of therapy in the patient (e.g., in consideration of including the course of therapy in the patient specific report 600), guides the course of therapy in the patient (e.g., in consideration of including analysis of projected outcomes from the course of therapy in the patient specific report 600), modifies the course of therapy in the patient (e.g., in consideration of including a modification of a dosage in the course of therapy in the patient specific report 600), or a combination thereof. For instance, in some embodiments, the allele detection system 200 determines the course of therapy in accordance with an evaluation of the gene-pharmaceutical composition lookup table 240 and/or the pharmaceutical composition-pharmaceutical composition lookup table 246 modules.

In some embodiments, the one or more neuropsychiatric disorder of the patient specific report 600 includes a depressive disorder (e.g., patient specific report 600-1 providing a “depression summary” report, patient specific report 600-2 providing an “depression augmentation summary” report, etc.), a bipolar disorder, an anxiety disorder, a post-traumatic stress disorder, a schizophrenia disorder, an attention deficit hyperactivity disorder (ADHD), an autism spectrum disorder, a treatment resistant form of one of the aforementioned disorders, or a combination thereof. For instance, in some embodiments, the patient specific report 600 provides information configured to one or more neuropsychiatric disorders that is associated with the patient (e.g., configured to a pre-existing condition of the patient, configured to one or more neuropsychiatric disorders associated with the determination of the allele status 242 (e.g., based on a result from genetic test result module 220 of FIG. 2). However, one skilled in the art will recognize that, while the present disclosure is described in terms of a patient specific report for a treatment of one or more neuropsychiatric disorders, the systems and methods disclosed herein are not limited thereto.

Each respective genomic loci in the plurality of genomic loci is associated with a therapeutic efficacy of a treatment, in a plurality of treatments (e.g., pharmaceutical composition association 244 of FIG. 2), for at least one neuropsychiatric disorder in the one or more neuropsychiatric disorders. Furthermore, each genomic loci is associated with one or more treatments of the at least one neuropsychiatric disorder. As such, a respective treatment can have a one-to-one relationship with a corresponding genomic loci or a one-to-many relationship. In some embodiments, a respective treatment includes a pharmaceutical composition 248 (e.g., a administering the pharmaceutical composition 248, block 510 of FIG. 5, etc.) or a class of pharmaceutical compositions 248. Furthermore, in some embodiments, a respective treatment includes a recommended deviation in lifestyle of the patient. In some embodiments, the recommended deviation in lifestyle of treatment includes a deviation in exercise conducted by the patient (e.g., data plot 612-2 of FIG. 6B representing a treatment for exercise). In some embodiments, the recommended deviation in lifestyle of treatment includes a deviation in smoking status of the patient (e.g., a treatment for associated with graphical icon 616-10 of FIG. 6B). In some embodiments, the recommended deviation in lifestyle of treatment includes a deviation in the personal hygiene of the patient. In some embodiments, the recommended deviation in lifestyle of treatment includes a deviation in a diet of the patient. In this way, a respective allele status for each genomic loci provides a determination as to one or more treatments and efficacy of the one or more treatments for at least one neuropsychiatric disorder.

In some embodiments, the plurality of genomic loci includes a locus from one or more human genes selected from the group consisting of CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, and MC4R. In some embodiments, the plurality of genomic loci includes at least one locus from each of the following human genes: CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, and MC4R.

In some embodiments, the plurality of genomic loci includes at least one locus corresponding to a SNP selected from the group consisting of rs7997012, rs6265, rs4680, rs2470890, rs2069514, rs35694136, rs2069526, rs762551, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs1135840, rs16947, rs1135824, rs35742686, rs3892097, rs5030655, rs5030867, rs5030865, rs5030656, rs1065852, rs5030863, rs5030862, rs5030865(T), rs774671100, rs28371706, rs61736512, rs1058164, rs59421388, rs28371725, rs35599367, rs776746, rs10264272, rs41303343, rs1061235, rs2395148, rs489693, rs1801131, rs1801133, rs25531, rs63749047, and rs1902023. In some embodiments, the plurality of genomic loci includes the loci corresponding to SNPs rs7997012, rs6265, rs4680, rs2470890, rs2069514, rs35694136, rs2069526, rs762551, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs1135840, rs16947, rs1135824, rs35742686, rs3892097, rs5030655, rs5030867, rs5030865, rs5030656, rs1065852, rs5030863, rs5030862, rs5030865(T), rs774671100, rs28371706, rs61736512, rs1058164, rs59421388, rs28371725, rs35599367, rs776746, rs10264272, rs41303343, rs1061235, rs2395148, rs489693, rs1801131, rs1801133, rs25531, rs63749047, and rs1902023.

In some embodiments, the plurality of genomic loci includes a locus from one or more human genes selected from the group consisting of CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, MC4R, 5HT2C, ADRA2A, ANK3, CACNA1C, DRD2, GRIK1, OPRM1, UGT1A4, and ABCB1. In some embodiments, the plurality of genomic loci includes at least one locus from each of the following human genes: CYP1A2, CYP2B6, CYP2C9, CYP2C19, CYP2D6, CYP3A4/5, SLC6A4, HTR2A, HLA-A, HLA-B, UGT2B15, MTHFR, BDNF, COMT, MC4R, 5HT2C, ADRA2A, ANK3, CACNA1C, DRD2, GRIK1, OPRM1, UGT1A4, and ABCB1.

In some embodiments, the plurality of genomic loci includes at least one locus corresponding to a SNP selected from the group consisting of rs7997012, rs3813929, rs1045642, rs2032583, rs1800544, rs10994336, rs6265, rs1006737, rs4680, rs2470890, rs2069514, rs35694136, rs2069526, rs762551, rs12720461, rs2069526, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs7900194, rs16947, rs1135840, rs1135824, rs35742686, rs3892097, rs5030655, rs5030867, rs5030865, rs5030656, rs1065852, rs5030863, rs5030862, rs5030865, rs774671100, rs28371706, rs16947, rs61736512, rs1058164, rs16947, rs59421388, rs1135840, rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023. In some embodiments, the plurality of genomic loci includes the loci corresponding to SNPs rs7997012, rs3813929, rs1045642, rs2032583, rs1800544, rs10994336, rs6265, rs1006737, rs4680, rs2470890, rs2069514, rs35694136, rs2069526, rs762551, rs12720461, rs2069526, rs72547513, rs2279343, rs3211371, rs3745274, rs2279343, rs4244285, rs17878459, rs4986893, rs57081121, rs28399504, rs56337013, rs72552267, rs72558186, rs41291556, rs17884712, rs6413438, rs12248560, rs12769205, rs3758581, rs1799853, rs1057910, rs56165452, rs28371686, rs9332131, rs7900194, rs28371685, rs72558187, rs7900194, rs16947, rs1135840, rs1135824, rs35742686, rs3892097, rs5030655, rs5030867, rs5030865, rs5030656, rs1065852, rs5030863, rs5030862, rs5030865, rs774671100, rs28371706, rs16947, rs61736512, rs1058164, rs16947, rs59421388, rs1135840, rs28371725, rs35599367, rs776746, rs10264272, rs41303343, rs1799732, rs2832407, rs1061235, rs2395148, rs489693, rs1801131, rs1801133, rs1799971, rs25531, rs63749047, rs2011425, and rs1902023.

In some embodiments, the plurality of genomic loci includes any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or all 82 of the genomic loci corresponding to a SNP selected from the group above. In some embodiments, the plurality of genomic loci includes at least three genomic loci, at least four genomic loci, at least five genomic loci, or at least 10 genomic loci in Table 1 and/or Table 2.

In some embodiments, these genomic loci and their SNP status may be evaluated in conjunction with other markers known in the art as associated with a therapeutic efficacy of treatment of a neuropsychiatric disorder. Exemplary types of the other markers include but are not limited to gene expression product, epigenetic modification of genomic DNA such as methylation, and metabolic profile/signature. Archer et al., (2010) “Epigenetics and Biomarkers in the Staging of Neuropsychiatric Disorders” Neurotox Res (2010) 18: 347; Quinones et al., “Metabolomics tools for identifying biomarkers for neuropsychiatric diseases,” (2009), Neurobiology of Disease, 35, (2) 165-176.

TABLE 1
Exemplary SNPs with known pharmacogenetic associations
244 for the treatment of one or more neuropsychiatric disorders.

| SNP | Position | Gene |
|---|---|---|
| rs7997012 | chr13:46837850 | HTR2A |
| rs3813929 | chr10:114584047 | HTR2C |
| rs1045642 | chr7:87509329 | ABCB1 |
| rs2032583 | chr7:87531245 | ABCB1 |
| rs1800544 | chr10:111076745 | ADRA2A |
| rs10994336 | chr10:60420054 | ANK3 |
| rs6265 | chr11:27658369 | BDNF |
| rs1006737 | chr12:2236129 | CACNA1C |
| rs4680 | chr22:19963748 | COMT |
| rs1799732 | chr11:113475530 | DRD2 |
| rs2832407 | chr21:29595188 | GRIK1 |
| rs489693 | chr18:60215554 | MC4R |
| rs1801131 | chr1:11794419 | MTHFR |
| rs1801133 | chr1:11796321 | MTHFR |
| rs1799971 | chr6:154039662 | OPRM1 |
| rs25531 | chr17:30237328 | SLC6A4 |
| rs63749047 | chr17:28564340 | SLC6A4 |

TABLE 2
Additional exemplary SNPs with known pharmacogenetic associations
244 for the treatment of neuropsychiatric disorders.

| SNP | Position | Gene |
|---|---|---|
| rs2470890 | chr15:74755085 | CYP1A2 |
| rs2069514 | chr15:74745879 | CYP1A2 |
| rs35694136 | chr15:74747272 | CYP1A2 |
| rs2069526 | chr15:74749000 | CYP1A2 |
| rs762551 | chr15:74749576 | CYP1A2 |
| rs72547513 | chr15:74750296 | CYP1A2 |
| rs2279343 | chr19:41009358 | CYP2B6 |
| rs3211371 | chr19:41016810 | CYP2B6 |
| rs3745274 | chr19:41006936 | CYP2B6 |
| rs2279343 | chr19:41009358 | CYP2B6 |
| rs4244285 | chr10:94781859 | CYP2C19 |
| rs17878459 | chr10:94775165 | CYP2C19 |
| rs4986893 | chr10:94780653 | CYP2C19 |
| rs57081121 | chr10:94780653 | CYP2C19 |
| rs28399504 | chr10:94762706 | CYP2C19 |
| rs56337013 | chr10:94852738 | CYP2C19 |
| rs72552267 | chr10:94775453 | CYP2C19 |
| rs72558186 | chr10:94781999 | CYP2C19 |
| rs41291556 | chr10:94775416 | CYP2C19 |
| rs17884712 | chr10:94775489 | CYP2C19 |
| rs6413438 | chr10:94781858 | CYP2C19 |
| rs12248560 | chr10:94761900 | CYP2C19 |
| rs12769205 | chr10:94775367 | CYP2C19 |
| rs3758581 | chr10:94842866 | CYP2C19 |
| rs1799853 | chr10:94942290 | CYP2C9 |
| rs1057910 | chr10:94981296 | CYP2C9 |
| rs56165452 | chr10:94981297 | CYP2C9 |
| rs28371686 | chr10:94981301 | CYP2C9 |
| rs9332131 | chr10:94949282 | CYP2C9 |
| rs7900194 | chr10:94942309 | CYP2C9 |
| rs28371685 | chr10:94981224 | CYP2C9 |
| rs72558187 | chr10:94941958 | CYP2C9 |
| rs1135840 | chr22:42126611 | CYP2D6 |
| rs16947 | chr22:42127941 | CYP2D6 |
| rs1135824 | chr22:42129042 | CYP2D6 |
| rs35742686 | chr22:42128242 | CYP2D6 |
| rs3892097 | chr22:421289454 | CYP2D6 |
| rs5030655 | chr22:42129084 | CYP2D6 |
| rs5030867 | chr22:42127856 | CYP2D6 |
| rs5030865 | chr22:42129033 | CYP2D6 |
| rs5030656 | chr22:42128174 | CYP2D6 |
| rs1065852 | chr22:42130692 | CYP2D6 |
| rs5030863 | chr22:42525912 | CYP2D6 |
| rs5030862 | chr22:42130668 | CYP2D6 |
| rs5030865 (T) | chr22:42129033 | CYP2D6 |
| rs774671100 | chr22:42130655 | CYP2D6 |
| rs28371706 | chr22:42129770 | CYP2D6 |
| rs61736512 | chr22:42129132 | CYP2D6 |
| rs1058164 | chr22:42129130 | CYP2D6 |
| rs59421388 | chr22:42127608 | CYP2D6 |
| rs28371725 | chr22:42127803 | CYP2D6 |
| rs35599367 | chr7:99768693 | CYP3A4 |
| rs776746 | chr7:99672916 | CYP3A5 |
| rs10264272 | chr7:99665212 | CYP3A5 |
| rs41303343 | chr7:99652771 | CYP3A5 |
| rs1061235 | chr6:29945521 | HLA-A |
| rs2395148 | chr6:32353777 | HLA-B |
| rs2011425 | chr2:233718962 | UGT1A4 |
| rs1902023 | chr4:68670366 | UGT2B15 |

Block 406. Referring to block 406, the method 400 includes displaying a graphical user interface on the display (e.g., display 308 of FIG. 3) of the client device 300. In this way, the graphical user interface provides a graphic visualization one or more treatments of the one or more neuropsychiatric disorders, which form a basis for the patient specific report 600.

The graphical user interface includes a respective graphical chart (e.g., graphical chart 602-1 of FIG. 6A, graphical chart 602-2 of FIG. 6B) for each corresponding neuropsychiatric disorder in the one or more neuropsychiatric disorders. In some embodiments, the patient specific report 600 includes two or more graphical charts 602 (e.g., a first graphical chart 602-1 of FIG. 6A and a second graphical chart 602-2 of FIG. 6B) for a corresponding neuropsychiatric disorder in the one or more in the one or more neuropsychiatric disorders of the patient specific report 600, such that additional information can be provided through at least the second graphical chart 602-2. For instance, in some embodiments, the at least two graphical charts 602 provide information that supplement the first graphical chart 602-1, augment the first graphical chart 602-1, is auxiliary too the first graphical chart 602-1, or a combination thereof. As a non-limiting example, a patient specific report 600 includes a first graphical chart 600-1 that provides a summary of an allele status 242 of a patient for a neuropsychiatric disorder of depression, and further includes a second graphical chart 602-2 that provides an augmentation to the summary of the allele status 242 of the patient for depression of the first graphical chart 602-1.

In some embodiments, the graphical user interface is restricted to displaying one respective graphical chart 602 at a time (e.g., limited by a display configuration of the respective graphical chart 602 and/or the client application 320 of the client device 300), allowing a subject to focus and comprehend the information provided by the respective graphical chart 602 without distraction of the second graphical chart 602-2. However, the present disclosure is not limited thereto.

In some embodiments, the displaying of the respective graphical chart 602 for each neuropsychiatric disorder includes displaying a plurality of respective graphical charts 602 of the one or more treatments. For instance, in some embodiments, the patient specific report 600 includes at least one respective graphical chart 602 for each neuropsychiatric disorder of the one or more neuropsychiatric disorders. For instance, in some embodiments, the patient specific report includes a respective graphical chart 602 for a corresponding neuropsychiatric disorder of a depression disorder (e.g., first graphical chart 602-1 of FIG. 6A, second graphical chart 602-2 of FIG. 6B), a bipolar disorder, an anxiety disorder, a post-traumatic stress disorder, a schizophrenia disorder, an attention deficit hyperactivity disorder (ADHD), an autism spectrum disorder, a treatment resistant form of one of the aforementioned disorders, or a combination thereof.

Each respective graphical chart 602 includes at least one axis (e.g., first axis 604-1 of FIG. 6A, second axis 604-2 of FIG. 6A, first axis 604-1 of FIG. 6B, second axis 604-2 of FIG. 6B, etc.). Each axis 604 is a feature on the respective graphical chart 602 that extends in a single direction along which information can be presented and/or conveyed through and defines a dimension of the respective graphical chart 602. That is, the position of a feature on the respective graphical chart 602 can be used to convey information with respect to the at least one axis 604. From this, each axis 604 allows the subject to visualize relative positions between features (e.g., data plots 612 of FIG. 6A, data plots 612 of FIG. 6B) on the respective graphical chart 602 with respect to a respective axis 604.

For instance, in some embodiments, each respective graphical chart 602 includes a first axis 604-1 and a second axis 604-2. In some embodiments, if a respective graphical chart 602 includes two or more axes 604, at least the first axis 604-1 is orthogonal to the second axis 604-2. For instance, as illustrated by the first graphical chart 600-1 of FIG. 6A, the first axis 604-1 and the second axis 604-2 intersect at a right angle, at substantially a right angle, or will intersect at said angles if either the first axis 604-1 or the second axis 604-2 were projected a further distance. In this way, the respective graphical chart 602 illustrates a quadrant formed by the first axis 604-1 and the second axis 604-2. However, the present disclosure is not limited thereto. As used here, the term “substantially” means within five degrees of a right angle.

In some embodiments, a respective axis 604 is displayed as a visible feature of the respective graphical chart 602. For instance, a respective second axis 604-2 of FIG. 6B is displayed as a bar shaped feature of the respective graphical chart. In some embodiments, the respective axis 604 is hidden feature of the respective graphical chart (e.g., respective first axis 604-1 of FIG. 6B).

In some embodiments, the information conveyed for a respective axis 604 is conveyed in a first dimension. In some embodiments, the information conveyed with for a respective axis 604 is conveyed in the first dimension and restricted to the first dimension. Accordingly, a feature of the graphical chart 602 (e.g., a frame of reference 610 of FIG. 6B, a data plot 612 of FIG. 6A, etc.) can convey information in accordance with a position of the feature with respect to the respective axis 604. However, in such embodiments, the position of the feature with respect to a second dimension does not convey information. As a non-limiting example, referring to the second graphical chart 602-2 of FIG. 6B, consider each position of a respective first axis 604-1, a respective second axis 604-2, and a respective feature of a second data plot 612-2. Here, the respective second axis 604-2 includes different magnitudes of one or more efficacies 803 for a treatment for a corresponding neuropsychiatric disorder of depression at respective boundaries of a corresponding first dimension, whereas the respective first axis 604-1 provides no information that corresponds to its respective boundaries of a corresponding first dimension (e.g., a second dimension of the respective second axis 604-2). Accordingly, the position of the second data plot 612-2 closer to a respective boundary of the respective second axis 604-2 conveys information related to efficacy associated with the respective boundary (e.g., the second data plot 612-2 is associated with a positive efficacy 608-3). On the other hand, the position of the second data plot 612-2 closer to a respective boundary of the respective first axis 604-1 does not convey information respect to the respective second boundary 604-2. However, the present disclosure is not limited thereto. For instance, referring to the first graphical chart 602-1 of FIG. 6A, in some embodiments, the position of the second data plot 612-2 closer to a respective boundary of the respective first axis 604-1 does convey information. In some embodiments, a first information convey by a position of a feature with respect to a respective first axis 604-1 is the same or different than a second information conveyed by the position of the feature with respect to a respective second axis 604-2.

In some embodiments, each respective first axis 604-1 is segmented into a respective plurality of bins (e.g., a plurality of bins 606 of FIG. 6A, a plurality of bins 606 of FIG. 6A). In some embodiments, each bin 606 in the respective plurality of bins 606 is associated with a respective independent first subset of genomic loci in the plurality of genomic loci. In some embodiments, this association with the respective independent first subset of genomic loci is formed through the gene-pharmaceutical composition lookup table 240. That is, in some embodiments, each respective bin 606 is associated with one or more allele status 242 of the gene-pharmaceutical composition lookup table 240. Additionally, each bin 606 in the respective plurality of bins 606 presents a respective treatment class in a plurality of treatment classes. For instance, referring to FIG. 6A, a respective first bin 606-1 represents a corresponding treatment class of SSRIs pharmaceutical compositions 248, whereas a respective second bin 606-2 represents a corresponding treatment class of SNRIs pharmaceutical compositions 248. From this, each bin 606 forms a link between the respective independent first subset of genomic loci and the respective treatment class through a corresponding bin 606.

As a non-limiting example, consider a determination of an allele status 242 for a patient including a first allele 242-1, a second allele status 242-2, and generating a patient specific report 600 for a first neuropsychiatric disorder (e.g., depression) based on the determination of the allele status 242. Further, consider the first allele status 242-1 including a first pharmaceutical composition association 244-1 of a first class of pharmaceutical compositions 248 and the first neuropsychiatric disorder, the second allele status 242-2 including a second pharmaceutical composition association 244-2 of a second class of pharmaceutical compositions 248 and a second neuropsychiatric disorder, and the third allele status 242-3 including a third pharmaceutical composition association 244-3 of a third class of pharmaceutical compositions 248 and the first neuropsychiatric disorder. Thus, when generating the patient specific report 600 for the first neuropsychiatric disorder a respective first graphical chart 602-1 includes a respective first bin 606-1 associated with the first allele status 242-1 and a respective second bin 606-2 associated with the third allele status 242-3. Furthermore, in some embodiments, the patient specific report 600 includes the first neuropsychiatric disorder and the second neuropsychiatric disorder, such a second respective graph 600-2 includes a respective thirds bin 606-3 associated with the second allele status 242-2.

Accordingly, the respective graphical chart 602 visualizes information for the subject that pertains to one or more treatment classes 602 and an efficacy 608 of the one or more treatment classes 602 that is formed through the association with the respective independent first subset of genomic loci. Moreover, this visualization occurs without a need to display information related to the respective independent first subset of genomic loci (e.g., information that identifies the respective independent first subset of genomic loci). In this way, the subject visualizing the patient specific report 600 can comprehend information related to a respective treatment class whilst also having an appreciation for the respective independent first subset of genomic loci associated with the respective treatment class, without having the mental burden of forming the linking association that is, instead, provided through the respective bin 606. Moreover, since each bin 606 represents a corresponding treatment class, the subject can now evaluate the corresponding neuropsychiatric disorder on a treatment class by treatment class basis.

In some embodiments, a respective bin 606 in the plurality of bins 606 represents a corresponding treatment class of a class of pharmaceutical compositions 248 of selective serotonin reuptake inhibitors (SSRIs) (e.g., first bin 606-1 of FIG. 6A), serotonin-norepinephrine reuptake inhibitors (SNRIs) (e.g., second bin 606-2 of FIG. 6A), tricyclic antidepressants (e.g., TCAs) (e.g., fourth bin 606-4 of FIG. 6A), or monoamine oxidase inhibitors (MAOIs). Accordingly, in some embodiments, each respective treatment in the one or more treatments of the respective treatment class is a corresponding pharmaceutical composition 248. For instance, in some embodiments, if the respective treatment class representing a respective bin 606 is SSRIs pharmaceutical compositions 248 (e.g., first bin 606-1 of FIG. 6A), the one or more treatments include a corresponding pharmaceutical composition 248 of Citalopram, Escitalopram, Fluoxetine, Paroxetine, and/or Sertraline, or a combination thereof. As another example, in some embodiments, if the respective treatment class representing a respective bin 606 is SNRIs pharmaceutical compositions 248 (e.g., second bin 606-2 of FIG. 6A), the one or more treatments include a corresponding pharmaceutical composition 248 of Desvenlafaxine, Duloxetine, Levomilnacipran, and/or Venlafaxine, or a combination thereof. As yet another example, in some embodiments, if the respective treatment class representing a respective bin 606 is TCAs pharmaceutical compositions 248 (e.g., fourth bin 606-4 of FIG. 6A), the one or more treatments include a corresponding pharmaceutical composition 248 of Amitriptyline, Amoxapine, Desipramine, Doxepin, Imipramine, Nortriptyline, Protriptyline, and/or Trimipramine, or a combination thereof.

In, some embodiments, each respective first axis 604-1 is segmented into 1, 2, 3, 4, 5, 6, 7, 8, 9, 10, 11, 12, 13, 14, 15, or 16 bins 606. As a non-limiting example, the first graphical chart 602-1 of FIG. 6A is segmented into a respective first bin 606-1, a respective second bin 606-2, a respective third bin 606-4, and a respective fourth bin 606-4. On the other hand, the second graphical chart 602-2 of FIG. 6B is segmented to contain a respective first bin 606-1. In some embodiments, a segmentation of each respective first axis 604-1 is based on a number of corresponding allele status 242 in the determination of the allele status 242 for the corresponding neuropsychiatric disorder of the respective graphical chart 602. For instance, if the determination of the allele status 242 for the corresponding neuropsychiatric disorder of the respective graphical chart 602 contains an respective allele status 242 with a plurality of pharmaceutical composition associations 244, the respective graphical chart is segmented into a plurality of bins 606 that equal in number to the a plurality of pharmaceutical composition associations 244 of the respective allele status 242. In some embodiments, the segmentation forms at least two abutting bins 606 that are displayed in different colors (e.g., a first color and a contrasting color). In some embodiments, a junction between the two abutting bins 606 is a feature of the respective graphical chart 602 that can convey information to the subject visualizing the patient specific report 600.

In some embodiments, the respective second axis 604-2 of the respective graphical chart 602 is associated with a parameter or a characteristic of an efficacy (e.g., efficacy 608 of FIG. 6A, efficacy 608 of FIG. 6B) of a treatment for the corresponding neuropsychiatric disorder that is associated with the respective graphical chart 602. In some embodiments, the parameter or the characteristic is a pharmacogenetic parameter, such as an allelic effect. For instance, in some embodiments, the parametric or the characteristic is a risk associated with the treatment. In this way, if the treatment is a corresponding pharmaceutical composition, an allelic effect on the corresponding pharmaceutical composition is visualized by a relative positioning with respect to the respective second axis 604-2 on the respective graphical chart 602. Additionally, in some embodiments, the parameter or the characteristic of the efficacy is a normalization of each unique parameter or characteristic of an efficacy for each feature of the respective graphical chart 602.

In some embodiments, each respective axis 604 defines a boundary of the respective graphical chart 602. For instance, in some embodiments, the respective first axis 604-1 defines one or more boundaries, such as a first upper boundary associated with the respective first axis 604-1, a first lower boundary associated with the respective first axis 604-1, or both. Moreover, in some embodiments, the respective second axis 604-4 defines one or more boundaries separate and apart from the one or more boundaries for the respective first axis 604-1. For instance, in some embodiments, a second upper boundary is associated with the respective second axis 604-2, a second lower boundary is associated with the respective second axis 604-2, or both. However, the present disclosure is not limited thereto. For instance, in some embodiments, the respective first axis 604-1 does not define a boundary of the respective graphical chart 602 whereas the respective second axis 604-2 defines at least one boundary of the respective graphical chart 602. In this way, the second respective axis 604-2 provides a reference the relative positions of one or more features of the respective graphical chart 602, while the respective first axis 604-1 provides the one or more features and/or aspects related to the respective second axis 604-2. Accordingly, each respective axis 604 can be associated with one or more aspects for a treatment of the one or more neuropsychiatric disorders.

In some embodiments, the respective second axis 604-2 includes a first boundary of the respective graphical chart 602. In some embodiments, the first boundary 608 is associated with one or more aspects of the one or more neuropsychiatric disorders, such as an efficacy (e.g., efficacy 608-2 of FIG. 6A, efficacy 608-3 of Figure of 6B) a treatment for the one or more neuropsychiatric disorders. For instance, in some embodiments, the second lower boundary of the respective second axis 604-2 is associated with a first efficacy 608-1 of the treatment and the second upper boundary of the respective second axis 604-2 is associated with a second efficacy 608-2 of the treatment. In some embodiments, the first efficacy 608-1 is a decrease in efficacy 608 of a treatment, an increase in efficacy 608 of the treatment, an unaffected efficacy 608 of the treatment, or an unknown effect on efficacy 608 of the treatment. However, the present disclosure is not limited thereto. In some embodiments, the first efficacy 608 is a first magnitude the first boundary is a largest detrimental change in efficacy 608 (e.g., efficacy 608-2 of FIG. 6A). In some embodiments, a second magnitude of a second boundary associated with a largest beneficial change in efficacy 608. In some embodiments, the first magnitude and the second magnitude are equal. For instance, referring briefly to FIG. 6A, a first efficacy 608-1 is associated with “Standard Options[,]” a second efficacy 608-2 is associated with “Alert/Caution[,]” and a third efficacy 608-3 is associated with “PGx Guided Options[.]”

In some embodiments, a respective first axis 604-1 of a first graphical chart 602-1 is the same as a respective first axis 604-1 of a second graphical chart 602-2. Moreover, in some embodiments, a respective second axis 604-2 of a first graphical chart 602-1 is the same as a respective second axis 604-2 of a second graphical chart 602-2. Furthermore, in some embodiments, a respective second axis 604-2 of a first graphical chart 602-1 is the same as a respective second axis 604-2 of a second graphical chart 602-2, whereas a respective first axis 604-1 of the first graphical chart 602-1 is different from a respective first axis 604-1 of the second graphical chart 602-2. From this, the respective second axis 604-2 remains a consistent feature between the first graphical chart 602-1 and the second graphical chart 602-2, which reduces a cognitive burden on a subject having to comprehend information from both graphical charts 602.

Each respective graphical chart 602 further includes one or more respective data plots (e.g., data plots 612 of FIG. 6A, data plots 612 of FIG. 6B, etc.). Each respective data plot 612 of the one or more respective data plots 612 is within the corresponding bin 606 that is associated with the corresponding treatment. For instance, referring to FIG. 6A, a first data plot 612-1 through a fifth data plot 616-5 are within the first bin 606-1, whereas a seventh data plot 612-7 is within a third bin 606-3 of the respective graphical chart 602-1. In addition, each respective data plot 612 is independently located relative to a position on the second axis 604-2. For instance, the first data plot 612-1 of FIG. 6A is in a first position interposing between a first efficacy 608-1 and a second efficacy 608-2, whereas a third data plot 612-3 is in a second position aligned with the efficacy 608-2, even though the first data plot 612-2 and the second data plot 612-2 are within the first bin 606-1. This position of each data plot 612 is based on the received allele status that was determined for the subject for the respective independent first subset of genomic loci associated with a corresponding bin 606. Each respective data plot 612 of the one or more respective data plots 612 represents a corresponding treatment in the plurality of treatments of the patient specific report 600. For instance, if each treatment in the plurality of treatments of the patient specific report 600 is a corresponding pharmaceutical composition 248, then the respective graphical chart 602 includes a respective data plot 612 for each corresponding pharmaceutical composition 248. As such, the therapeutic efficacy of the corresponding treatment is provided through a position of the respective data plot 612 of the respective graphical chart 602. Accordingly, the subject can visualizing one or more treatments of the one or more neuropsychiatric disorders by viewing the positioning of the one or more data plots 612 displayed through the patient specific report 600.

In some embodiments, a respective bin 606 represents a homogenous treatment class, such as a treatment class of pharmaceutical compositions 248 having a homogenous characteristic. For instance, in some embodiments, a respective bin 606 in the respective plurality of bins 606 represents a treatment class of SSRIs pharmaceutical compositions 248. Accordingly, each respective treatment in the one or more treatments is a corresponding pharmaceutical composition 248 belonging to the SSRIs class (e.g., first bin 606-1 of FIG. 6A). As such, in some embodiments, the respective bin 606 includes a data plot for Citalopram (e.g., first data plot 612-1 of FIG. 6A), Escitalopram (e.g., second data plot 612-2 of FIG. 6A), Fluoxetine (e.g., third data plot 612-3 of FIG. 6A), Paroxetine (e.g., fourth data plot 612-4 of FIG. 6A), and/or Sertraline (e.g., fifth data plot 612-5 of FIG. 6A), or a combination thereof. As a non-limiting example, consider a respective first bin 606-1 that represents a first treatment class of pharmaceutical compositions 248, a determination of an allele status for a first allele status 242-1 of the patient, and the first allele status 242-2 includes a first pharmaceutical composition association 244-1 that represents the first treatment class of pharmaceutical compositions 248. Accordingly, based on an evaluation of the gene-pharmaceutical composition lookup table 240 and, optionally, the pharmaceutical composition-pharmaceutical composition lookup table 246, the system 100 includes a respective data plot 612 within the respective first bin 606-1 for one or more pharmaceutical compositions 248 that is a SSRIs pharmaceutical composition 248.

As another non-limiting example, referring to FIG. 6A, the respective graphical chart 602 includes a respective first bin 606-1 that represents a treatment class of SSRIs pharmaceutical compositions 248. Accordingly, each respective data plot 612 within the respective first bin 606-1 is a SSRIs pharmaceutical composition 248. As such, respective first bin 606-1 of the respective graphical chart 602 includes a first data plot 612-1 that represents a Citalopram pharmaceutical composition 248, a second data plot 612-2 that represents a Escitalopram pharmaceutical composition 248, a third data plot 612-3 that represents a Fluoxetine pharmaceutical composition 248, a fourth data plot 612-4 that represents a Paroxetine pharmaceutical composition 248, and a fifth data plot 612-5 that represents a Sertraline pharmaceutical composition 248. Moreover, each of the first data plot 612-1 through the fifth data plot 612-5 of FIG. 6A forms a respective first cluster 618-1 of data plots 612. From this, the subject visualizing the respective graphical chart 602 can comprehend the efficacy 608 of the SSRIs treatment class of pharmaceutical compositions 608 identified above based on the relative location of each corresponding data plot 612. Moreover, since each of the corresponding data plots 612 is within the respective first bin 606-1, the subject can comprehend the efficacy 608 of the SSRIs treatment class by visualizing the respective first cluster 618-1 of data plots 612 formed through the first respective bin 606-1.

As yet another non-limiting example, in some embodiments, a respective bin 606 in the respective plurality of bins 606 represents a treatment class of SNRIs (e.g., second bin 606-2 of FIG. 6A that represents a treatment class of SNRIs pharmaceutical compositions 248). Accordingly, the respective bin 606 includes a respective data plot 612 for pharmaceutical compositions 248 including Desvenlafaxine, Duloxetine, Levomilnacipran, and/or Venlafaxine, or a combination thereof (e.g., second bin 606-2 includes a corresponding data plot 612 that represents Desvenlafaxine, a corresponding data plot 612-6 that represents Duloxetine, a corresponding data plot 612 that represents Levomilnacipran, and/or a corresponding data plot 612 that represents Venlafaxine, or a combination thereof).

As yet another non-limiting example, in some embodiments, a respective bin 606 in the respective plurality of bins 606 represents a treatment class of TCAs (e.g., fourth bin 606-4 of FIG. 6A that represents a treatment class of TCAs pharmaceutical compositions 248). Accordingly, the respective bin 606 includes a respective data plot 612 for pharmaceutical compositions 248 including Amitriptyline, Amoxapine, Desipramine, Doxepin, Imipramine, Nortriptyline, Protriptyline, and/or Trimipramine, or a combination thereof (e.g., fourth bin 606-4 includes a corresponding data plot 612 that represents Amitriptyline, a corresponding data plot 612-6 that represents Amoxapine, a corresponding data plot 612 that represents Desipramine, a corresponding data plot 612 that represents Doxepin, a corresponding data plot 612 that represents Imipramine, a corresponding data plot 612-8 that represents Nortriptyline, a corresponding data plot 612 that represents Protriptyline, and/or a corresponding data plot 612 that represents Trimipramine, or a combination thereof).

As yet another non-limiting example, in some embodiments, a respective bin 606 in the respective plurality of bins 606 represents a treatment class of one or more treatments with a homogenous characteristic (e.g., third bin 606-3 of FIG. 6A that represents a treatment class of “Other” that has a homogenous characteristic of being a treatment for the neuropsychiatric disorder of depression). Accordingly, the respective bin 606 includes a respective data plot 612 for pharmaceutical compositions 248 including Bupropion, Mirtazapine, Nefazodone, Trazodone, Vilazodone, Vortioxetine, or a combination thereof (e.g., third bin 606-3 includes a corresponding data plot 612 that represents Bupropion, a corresponding data plot 612-6 that represents Amoxapine, a corresponding data plot 612 that represents Mirtazapine, a corresponding data plot 612 that represents Nefazodone, a corresponding data plot 612 that represents Trazodone, a corresponding data plot 612-8 that represents Vilazodone, and/or a corresponding data plot 612 that represents Vortioxetine, or a combination thereof).

However, the present disclosure is not limited thereto. For instance, in some embodiments, a respective bin 606 represents a heterogeneous treatment class. In some embodiments, the heterogeneous treatment class includes a plurality of pharmaceutical compositions 248 that contain a respective pharmaceutical composition 248 from at least two classes of pharmaceutical compositions 248, such that the heterogeneous characteristic is a diversity in class of pharmaceutical composition 248. As a non-limiting ex example, in some embodiments, the respective third bin 606-3 of FIG. 6A that represents a treatment class of “Other” includes one or more data plots 612 that represent a corresponding plurality of pharmaceutical composition 248 that do not belong to a corresponding treatment class of a respective bin 606 other than the respective third bin 606-3. However, the present disclosure is not limited thereto. In some embodiment, a respective data plot 612 that represent a corresponding treatment is unassociated with a bin 606 in the respective plurality of bins 606. For instance, in other embodiments, the data plots 612 of the respective third bin 606-3 of FIG. 6A that represents a treatment class of “Other,” are unassociated with a respective bin 606 other than the respective third bin 606-3.

In some embodiments, each respective data plot 612 consumes a corresponding region of the respective graphical chart 602 (e.g., a corresponding area of the respective graphical chart 602). In some embodiments, the area of the region consumed by each respective data plot 612 is the same, such that each respective data plot 612 is displayed with a uniform shape. For instance, FIGS. 6A and 6B depict the respective data plots 612 of the respective graphical charts 602 consuming corresponding rectangular regions of the respective graphical chart 602 of equal area. In some embodiments, the area of the region consumed by each respective data plot 612 is associated with a variability in the efficacy 608.

In some embodiments, the respective region of a respective data plot 612 includes a text portion (e.g., first text portion 614-1 of FIG. 6A, first text portion 614-1 of FIG. 6A, third text portion 614-3 of FIG. 6B, etc.) that provides text based information related to the respective data plot 612, such as an identification of the pharmaceutical composition of the corresponding treatment of the respective data plot 612. The text portion 614 can be contained within the area of the respective region or outside of the respective region. For instance, referring to FIG. 6B, the respective first text region 614-1 provides an identification of “Olanzapine/Fluoxetine” pharmaceutical composition contained in the respective first data plot 602-1. In some embodiments, the identification includes a class of the corresponding pharmaceutical composition (e.g., a treatment class of the respective data plot 612). In some embodiments, a color of the text portion 614 is based on a feature of the respective data plot 612 (e.g., a color of the respective data plot 612).

In some embodiments, each respective genomic loci in a respective first subset of genomic loci of a bin 606 of the respective graphical chart 602 is associated with at least one respective data plot 612 of the one or more respective data plots 612. Furthermore, in some embodiments, each respective data plot 612 that is within a respective first bin 606-1 in the respective plurality of bins 606 forms a corresponding cluster of data plots (e.g., first cluster 618-1 of FIG. 6A, second cluster 618-2 of FIG. 6A, etc.) within the bin 606. Furthermore, in some embodiments, the respective first bin 606-1 is associated with a unique location on the respective first axis 604-1. In this way, adjacent bins 606 are formed with non-overlapping boundaries, such that a first cluster 618-1 of a first bin 606-1 does not overlap a second cluster 618-2 of a second bin 606-2. In some embodiments, the corresponding cluster 618 includes a visual feature on the respective graphical chart 602, such as a outline surrounding each data plot 612 within the respective bin 606.

In some embodiments, the respective graphical chart 602 includes one or more frames of reference (e.g., first frame of reference 610-1 of FIG. 6A, second frame of reference 610-2 of FIG. 6B, third frame of reference 610-3 of FIG. 6A, etc.). In some embodiments, a frame of reference 610 includes a demarcation that identifies the frame of reference 610 on the respective graphical chart 602. For instance, in some embodiments, the demarcation of the frame of reference 610 is displayed perpendicular to the second axis 604-2. As an example, referring to FIG. 6A, a first frame of reference 610-1 and a third frame of reference 610-3 each include a corresponding demarcation that is displayed as a solid line perpendicular to the second axis 604-2. From this, each frame of reference 610 provides a higher degree of accuracy and precision when discerning relative positioning of features (e.g., the data plots 612 with respect to the respective second axis 604-2 of the respective graphical chart 602 based on the frame of references 610.

In some embodiments, a first frame of reference 610-1 is associated with a first therapeutic efficacy 608-1 of the second axis 604-2. In some embodiments, the first therapeutic efficacy 608-1 is an equilibrium efficacy 608, such as an efficacy 608 on a normalized patient. For instance, in some embodiments, the equilibrium efficacy 608 is a standard efficacy 608 as determined by one of skill in the art. As an example, in some embodiments, the equilibrium efficacy 608 associated with the first frame of reference 610-1 is an effect on the patient in response to administering a treatment. In this way, a relative position of a respective data plot 612 with respect to the first frame of reference 610 provides information related to if a genomic loci and/or treatment associated with the respective data plot 612 is associated with a positive response (e.g., a relative position of the respective data plot 612 with respect to the first frame of reference 610 in a first direction, such as towards a first boundary of the respective second axis 604-2), a negative response (e.g., a relative position of the respective data plot 612 with respect to the first frame of reference 610-1 in a second direction, such as away from the first boundary of the respective second axis 604-2), or no response.

In this way, in some embodiments, a distance from the frame of reference 610 to the location of a respective data plot 612 (e.g., first distance D1 of FIG. 6B, second distance D2 of FIG. 6B) corresponds to a deviation in efficacy 608 of the corresponding treatment relative to the first efficacy 608-1 of the frame of reference 610. As such, greater distances from the frame of reference 610 is based on an amount of the deviation in efficacy 608 of the corresponding treatment. In this way, in some embodiments, the distance from the frame of reference 610 is either in a first direction or a second direction, allowing the deviation in efficacy 608 to be represent a positive deviation in efficacy 608 (e.g., distance D2 of FIG. 6B) or a second direction indicating a negative deviation in efficacy 608 (e.g., distance D1 of FIG. 6B). In some embodiments, the first direction opposes the second direction (e.g., the first direction is towards a left hand side of the respective graphical chart 602 and the second direction is towards a right hand side of the respective graphical chart 602).

As an example, referring briefly to FIG. 6B, consider a respective first data plot 612-1, a respective second data plot 612-2, and a respective third data plot 612-3 with a respective first frame of reference 610-1. Here, the respective first data plot 612-1 represents a corresponding treatment of the pharmaceutical composition 248 of Esketamine, the respective second data plot 612-2 represents a corresponding treatment of Exercise, and the respective third data plot 612-3 represents a corresponding treatment of the pharmaceutical compositions 248 of Olanzapine/Fluoxetine. The respective graphical chart 602-2 of FIG. 6B depicts the corresponding the respective first data plot 612-1 separated from the respective first frame of reference 610-1 by a first distance D1. This first distance D1 is in a first direction (e.g., towards a respective first axis 604-1) that indicates a negative deviation in the efficacy 608-2. Moreover, the respective graphical chart 602-2 depicts the respective second data plot 612-2 separated from the respective first frame of reference 610-1 by a second distance D2. Similar to the first distance D1, this second distance D2 is in a second direction (e.g., away from the respective first axis 604-1) that indicates a positive deviation in efficacy 608-3. Furthermore, the respective third data plot 612-3 is depicted straddling the respective first frame of reference 610-1, such that the centroid of the respective third data plot 612-3 intersects the respective first frame of reference 610-1 (e.g., a distance from the respective third data plot 612-3 to the respective first frame of reference 610-1 is zero). From this, the subject visualizing the respective graphical chart 602 of FIG. 6B can comprehend that the patient of the patient specific report 600 will have a negative response to the treatment of the pharmaceutical compositions 248 of Olanzapine/Fluoxetine for the neuropsychiatric disorder of depression based on a relative positioning of the respective first data plot 612-1 with respective to respective first frame of reference 610-1. Moreover, the patient of the patient specific report 600 will have a positive response to the treatment of Exercise for the neuropsychiatric disorder of depression based on a relative positioning of the respective second data plot 612-2 with respective to respective first frame of reference 610-1. Furthermore, the patient of the patient specific report 600 will have no change in response in comparison to a baseline response to the treatment of the pharmaceutical composition 248 of Esketamine for the neuropsychiatric disorder of depression based on a relative positioning of the respective third data plot 612-3 with respective to respective first frame of reference 610-1. Additionally, since the first distance D1 is greater than the second distance D2, the subject can comprehend that the negative response associated with the treatment of the pharmaceutical compositions 248 of Olanzapine/Fluoxetine is more severe (e.g., a greater magnitude) than the positive response associated with the treatment of Exercise. In this way, when the first distance and the second distance are in the same direction (e.g., both in the first direction or the second direction), the magnitude of the first and second distances is related to a degree of response in the efficacy 608 of the treatment, such that the subject can recommend and/or administer a corresponding treatment having a greater or lesser magnitude.

In some embodiments, a relation between the distance from the frame of reference 610 and the deviation in efficacy 608 is a linear relation. Accordingly, a first ratio of respective distances from the frame of reference 610 is linearly proportional, such that a second ratio of the magnitudes in efficacy 608 is the same. For instance, if a respective first ratio of the first distance D1 and the second distance D2 of FIG. 6B is 2:1, then a respective second ratio of the magnitudes of efficacy 608 of the corresponding treatments of the first data plot 612-1 and the second data plot 612-2 is 2:1. However, the present disclosure is not limited thereto. In some embodiments, the relation between the distance from the frame of reference 610 and the deviation in efficacy 608 is a monotonic function relation (e.g., a square root function relation or a logarithmic function relation). For instance, with the square root function relation, if the respective first ratio of the first distance D1 and the second distance D2 of FIG. 6B is 2:1, then the respective second ratio of the magnitudes of efficacy 608 of the corresponding treatments of the first data plot 612-1 and the second data plot 612-2 is 4:1 (e.g., efficacy 608 in administering Olanzapine/Fluoxetine to the subject in comparison to Exercise). Furthermore, in some embodiments, a relation between the distance from the frame of reference 610 and the deviation in efficacy 608 is a logarithmic relation. Thus, the first ratio of respective distances from the frame of reference 610 is proportional to log(N), with any logarithmic base, proportional to log(N+c), or proportional to c+log(N), where c is a constant, which can be a positive number, such as 1, and N is a positive integer. Also, in some embodiments, a blended function can also be used. For instance, in some embodiments, the relation between the distance from the frame of reference 610 and the deviation in efficacy 608 is different for different ranges of N As a non-limiting example, in some embodiments, the relation between the distance from the frame of reference 610 and the deviation in efficacy 608 is: (i) proportional or approximately proportional to N for values of N that are below a first threshold value of N; (ii) proportional, or approximately proportional, to the square-root of N over an intermediate range of values of N that is greater than or equal to the first threshold value of N and less than or equal to a second threshold value of N; and (iii) proportional, or approximately proportional, to log(N) above the second threshold value of N. However, the present disclosure is not limited thereto.

Additionally, in some embodiments, the boundary of the respective graphical chart 602 is determined by a floor function and/or a ceiling function. Accordingly, the distance from the frame of reference 610 to a respective data plot 612 is never less than a minimum floor value and/or never exceeds a maximum ceiling value.

In some embodiments, each therapeutic efficacy 608 is associated with a corresponding hue, such as a first therapeutic efficacy 608-1 of the respective second axis 602-4 is associated with a first hue. Accordingly, the relative distance from the frame of reference 610 to each respective data plot 612 shifts a color of the respective data plot 612 from the first hue to a second hue. However, the present disclosure is not limited thereto. For instance, in some embodiments, the shift in color is from a first tine to a second tint, from a first tone to a second tone, from a first shade to a second shade, or a combination thereof. Nevertheless, in some embodiments, this shift is in accordance with a function of the relative distance, such as one or more of the functions of the relation between the distance from the frame of reference 610 and the deviation in efficacy 608 described supra. In some embodiments, the frame of reference 610 forms a junction between two abutting features of the respective graphical chart 602, in which the two abutting features are displayed in different colors. As such, the junction formed by the frame of reference 610 conveys information to the subject with respect to the two features.

In some embodiments, the respective second axis 604-2 includes a first boundary associated with a first magnitude of a largest detrimental change in efficacy 608 (e.g., second efficacy 608-2 associated with an “Alert/Caution” of FIG. 6A) and a second magnitude of a second boundary associated with a largest beneficial change in efficacy 608 (e.g., third efficacy 608-3 associated with “PGx Guided Options” of FIG. 6A). In some embodiments, each efficacy 608 of the first magnitude and the second magnitude are equal.

For a subset of data plots 612 of the one or more respective data plots 612 of the respective graphical chart 602, each respective region of a corresponding data plot 612 includes one or more graphical icons in a plurality of graphical icons (e.g., graphical icons 616 of FIG. 6A, graphical icons 616 of FIG. 6B, etc.).

The subset of data plots 612 can include each respective data plots 612 or a portion that is less than all of the of one or more respective data plots 612 of the respective graphical chart 602. For instance, in some embodiments, the subset of data plots 612 is selected based on one or more threshold criteria. As an example, in some embodiments, the subset of data plots 612 includes each data plot 612 of the one or more respective data plots 612 that satisfies a threshold parameter or characteristic of a respective efficacy 608. In some embodiments, the threshold parameter or characteristic includes a first threshold magnitude as a function of the first magnitude, a second threshold magnitude as a function of the second magnitude, a threshold parameter or characteristic associated with the subject, or a threshold parameter or characteristic associated with a respective pharmaceutical composition 248.

Accordingly, the subset of data plots 612 each include a respective graphical icon 616 in the plurality of graphical icons 616 that represents a parameter or characteristic of the corresponding treatment represented by the corresponding data plot 612. From this, the patient specific report 600 provides information to the subject regarding a reason for the change in efficacy 608 of the corresponding treatment through the use of one or more graphical icons 616 associated with the corresponding data plot 612. For instance, in some embodiments, in accordance with a determination that a change in serum levels (e.g., exposure with a pharmaceutical composition 248) causes a change in deviation, a respective graphical icon 616 that represents the change in serum level is displayed, allowing the subject to comprehend a reasoning as to an underlying mechanism for the change in deviation.

Furthermore, in some embodiments, each graphical icon 616 includes a unique shape that relates to the parameter or characteristic represented by the respective graphical icon 616. For instance, in some embodiments, each respective graphical icon 616 is uniquely visually discernable marker or feature that conveys information to the subject visualizing the patient specific report 600. In some embodiments, a respective graphical icon is a filled shaped (e.g., filled with one or more colors), an unfilled shape. Shapes include a circle, a polygon, or other shape, such as arrow (e.g., “↑”, graphical icon 616-9 of FIG. 6B, graphical icon 606-9a of FIG. 6B, etc.). In some embodiments, a respective graphical icon 616 is a group of pixels represented in one or more colors that is different from a color of an adjacent pixel. In some embodiments, a respective graphical icon 616 is a text label (e.g., graphical icon 616-3 of FIG. 6A including text label “[1]” that represents a prodrug pharmaceutical composition class). In some embodiments, a respective graphical icon 616 is an image (e.g., graphical icon 616-1 of FIG. 6B including an image of a scale).

In some embodiments, the respective graphical chart includes a figure legend 620 illustrating a subset of the plurality of graphical icons 616. In some embodiments, the figure legend 620 is displayed beneath the respective first axis 604-1 and the respective second axis 604-2, such as the figure legend 620 of FIG. 6B. In some embodiments, the figure legend 620 is displayed adjacent to either the respective first axis 604-1 or the respective second axis 604-2. In some embodiments, the subset of graphical icons 616 of the figure legend 620 contains each graphical icon 616 in the plurality of graphical icons 616. For instance, if a respective graphical icon 616 is not associated with a data plot 612 of the respective graphical chart 620, in some embodiments, the figure legend 620 excludes the respective graphical icon 616.

In some embodiments, each respective graphical icon 616 in the plurality of graphical icons 616 is provided a corresponding rank. In some embodiments, a corresponding rank of a respective graphical icon 616 is determined in accordance with a weight (e.g., significance) of the graphical icon in providing a change in efficacy 608 of the corresponding treatment. For instance, if a first characteristic associated with a first graphical icon 616-1 provides a first deviation in efficacy 608 for a first data plot 612-1 and a second graphical icon 616-2 provides a second deviation in efficacy 608 for the data plot 612-1 that is less than the first deviation of the first graphical icon 616-1, than the first graphical icon 616-1 is ranked higher than or less than the second graphical icon 616-2. Furthermore, in some embodiments, each respective graphical icon 616 of a respective region of a corresponding data plot 612 is displayed in an ordered form by rank, such as a highest ranked graphical icon 616 being displayed closest to, or furthest from, an edge portion of the respective region.

In some embodiments, the plurality of graphical icons 616 includes a respective first graphical icon 616 that represents a change in weight of the patient (e.g., first graphical icon 616-1 of FIG. 6B). This change in weight of the patient can be a specific change in weight, such as a “Weight Gain” of the respective first graphical icon 616-1 of FIG. 6B, or a general change in weight. In some embodiments, the respective first graphical icon 616-1 is associated with a second generation antipsychotic pharmaceutical composition 248 (e.g., a class of treatment including a class of second generation antipsychotic pharmaceutical compositions 248). Accordingly, in some embodiments, in accordance with a determination that a corresponding pharmaceutical composition 248 of a respective data plot 612 belongs to the second generation antipsychotic pharmaceutical composition 248 class, the corresponding region of the respective data plot 612 includes the respective first graphical icon 616-1. In some embodiments, in accordance with a determination of an allele status 242 of the patient for the MC4R genomic loci (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective first graphical icon 616-1.

In some embodiments, the plurality of graphical icons 616 includes a respective second graphical icon 616 that represents a deviation in efficacy 608 based on a characteristic of the patient (e.g., second graphical icon 616-2 of FIG. 6B). In some embodiments, the characteristic of the patient includes a race of the patient and/or an ethnic characteristic of the patient. In some embodiments, the respective second graphical icon 616 is associated with a brain-derived neurotrophic factor antidepressant pharmaceutical composition 248 (e.g., a class of treatment including a class of brain-derived neurotrophic factor pharmaceutical compositions 248). Accordingly, in some embodiments, in accordance with a determination that a corresponding pharmaceutical composition of a respective data plot 612 belongs to the brain-derived neurotrophic factor pharmaceutical composition 248 class, the corresponding region of the respective data plot 612 includes the respective second graphical icon 616-2. In some embodiments, in accordance with a determination of a race of the patient and/or an ethnic characteristic of the patient, the corresponding region of the respective data plot 612 includes the respective second graphical icon 616-2.

In some embodiments, the plurality of graphical icons 616 incudes a respective graphical icon 616 that represents a deviation in efficacy 608 of a pharmaceutical composition 248, such as a respective graphical icon 616-3 representing a positive deviation in efficacy 608 of the pharmaceutical composition 248 (e.g., graphical icon 616-3 of FIG. 6B) and/or a respective fourth graphical icon 616-4 representing a negative deviation in efficacy 608 of the pharmaceutical composition 248 (e.g., graphical icon 616-4 of FIG. 6B). Furthermore, in some embodiments, the pharmaceutical composition 248 of the respective third graphical icon 616-3 and/or the respective fourth graphical icon 616-4 is different from the corresponding pharmaceutical composition 248 of the respective data plot 612. In this way, the respective third graphical icon 616-3 and/or the respective fourth graphical icon 616-4 provide information relating to an effect of co-administration the corresponding pharmaceutical composition 248 of the respective data plot 612 with respect to the pharmaceutical composition 248 of the respective third graphical icon 616-3 and/or the respective fourth graphical icon 616-4. However, the present disclosure is not limited thereto.

In some embodiments, the respective third graphical icon 616-3 and the respective fourth graphical icon 616-4 are each respectively associated with an antidepressant pharmaceutical composition 248 (e.g., a class of treatment including a class of antidepressant pharmaceutical compositions 248), an attention deficit hyperactivity disorder pharmaceutical composition 248 (e.g., a class of treatment including a class of ADHD pharmaceutical compositions 248), or an antipsychotic pharmaceutical composition 248 (e.g., a class of treatment including a class of antipsychotic pharmaceutical compositions 248), or a combination thereof. In some embodiments, the respective third graphical icon 616-3 and the respective fourth graphical icon 616-4 are each respectively associated with an antidepressant pharmaceutical composition 248 or an antipsychotic pharmaceutical composition 248 (e.g., a class of treatment including a class of antipsychotic pharmaceutical compositions 248 and/or a class of antidepressant pharmaceutical composition 248). As an example, in some embodiments, in accordance with a determination that a corresponding pharmaceutical composition 248 of a respective data plot 612 belongs to the antipsychotic pharmaceutical composition 248 class and/or the antidepressant pharmaceutical composition 248 class (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective third graphical icon 616-3 and/or the respective fourth graphical icon 616-4, accordingly.

In some embodiments, the plurality of graphical icons 616 incudes a respective graphical icon 616 that represents a deviation in tolerance with consumption (e.g., administration) of a pharmaceutical composition 248, such as a respective fifth graphical icon 616-5 representing a positive deviation in tolerance of the pharmaceutical composition 248 (e.g., graphical icon 616-5 of FIG. 6B) and/or a respective sixth graphical icon 616-6 representing a negative deviation in tolerance of the pharmaceutical composition 248 (e.g., graphical icon 616-4 of FIG. 6B). Furthermore, in some embodiments, the pharmaceutical composition 248 of the respective fifth graphical icon 616-5 and/or the respective sixth graphical icon 616-6 is different from the corresponding pharmaceutical composition 248 of the respective data plot 612. In this way, the respective fifth graphical icon 616-5 and/or the respective sixth graphical icon 616-6 provide information relating to an effect of co-administering the corresponding pharmaceutical composition 248 of the respective data plot 612 with respect to the pharmaceutical composition 248 of the respective fifth graphical icon 616-5 and/or the respective sixth graphical icon 616-6. However, the present disclosure is not limited thereto. In some embodiments, the respective fifth graphical icon 616-5 and/or the respective sixth graphical icon 616-6 are each respectively associated with an opioid pharmaceutical composition 248 (e.g., a class of treatment including a class of opioid pharmaceutical compositions 248). Accordingly, in some embodiments, in accordance with a determination that a corresponding pharmaceutical composition 248 of a respective data plot 612 belongs to the opioid pharmaceutical composition 248 class (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective fifth graphical icon 616-5 and/or the respective sixth graphical icon 616-6.

In some embodiments, the plurality of graphical icons 616 includes a respective graphical icon 616 that represents an adverse side effect with consumption of a pharmaceutical composition 248 (e.g., seventh graphical icon 616-7 of FIG. 6B). In some embodiments, in accordance with a determine that an adverse side effect threshold risk is satisfied, the corresponding region of the respective data plot 612 includes the seventh graphical icon 616-7. In some embodiments, the adverse side effect threshold risk is uniform for each patient, such as a uniform threshold risk of 50% chance of occurrence of the adverse side effect. In other embodiments, the adverse side effect threshold risk is specific to a respective patient. For instance, in some embodiments, the adverse side effect threshold risk is determined based on one or more characteristics are the patient.

In some embodiments, the plurality of graphical icons 616 includes a respective graphical icon 616 that represents a warning associated with consumption of a pharmaceutical composition 248 (e.g., eighth graphical icon 616-8 of FIG. 6B). In some embodiments, the respective eighth graphical icon 616-8 is associated with an anticonvulsant pharmaceutical composition 248 (e.g., a class of treatment including a class of anticonvulsant pharmaceutical compositions 248). Accordingly, in some embodiments, in accordance with a determination that a corresponding pharmaceutical composition of a respective data plot 612 belongs to the anticonvulsant pharmaceutical composition 248 class (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective eighth graphical icon 616-8.

In some embodiments, the plurality of graphical icons 616 includes a respective graphical icon 616 that represents a deviation in exposure with consumption of a pharmaceutical composition 248 (e.g., graphical icon 616-9 of FIG. 6B). For instance, in some embodiments, a respective ninth graphical icon 616-9 represents a positive deviation in exposure with the pharmaceutical composition 248 (e.g., graphical icon 616-9a of FIG. 6B) and/or a respective tenth graphical icon 616-10 represents a negative deviation in exposure with the pharmaceutical composition 248.

In some embodiments, the plurality of graphical icons 616 includes a respective graphical icon 616 that represents a deviation in exposure with consumption of a cytochrome P450 1A2 inhibitor pharmaceutical composition 248 (e.g., graphical icon 616-10 of FIG. 6B). In the embodiments, the deviation in exposure with consumption of the cytochrome P450 1A2 inhibitor pharmaceutical composition 248 is a negative deviation. In some embodiments, in accordance with a determination that a corresponding pharmaceutical composition of a respective data plot 612 is the cytochrome P450 1A2 inhibitor pharmaceutical composition 248 (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective eleventh graphical icon 616-11.

In some embodiments, the plurality of graphical icons 616 includes a respective graphical icon 616 that represents a prodrug pharmaceutical composition 248 (e.g., a class of treatment including a class of prodrug pharmaceutical compositions 248), such as a respective twelfth graphical icon 616-12 of FIG. 6B. Accordingly, in some embodiments, in accordance with a determination that a corresponding pharmaceutical composition 248 of a respective data plot 612 belongs to the prodrug pharmaceutical composition 248 class (e.g., based on an evaluation of the gene-pharmaceutical composition lookup table 240 and/or the pharmaceutical composition-pharmaceutical composition lookup table 246 of FIG. 2), the corresponding region of the respective data plot 612 includes the respective twelfth graphical icon 616-2.

In some embodiments, a respective data plot 612 and/or a respective graphical icon 616 is an intractable feature displayed by the graphical user interface. For instance, in some embodiments, in accordance with a user interaction with the respective data plot 612 or the respective graphical icon 616, the patient specific report 600 provides information that supplements the respective data plot 612 or the respective graphical icon 616, augments the respective data plot 612 or the respective graphical icon 616, is auxiliary too the respective data plot 612 or the respective graphical icon 616, or a combination thereof.

Now that methods 400 for generating and displaying a patient specific report 600 have has been described, methods 500 for generating and displaying a patient specific report 600 will be described in conjunction with FIG. 5.

Block 502. Referring to block 502 of FIG. 5, a method 500 includes a system (e.g., system 100 of FIG. 1, system of method 400 of FIG. 4, etc.) for displaying a patient specific report (e.g., patient specific report 600 of method 400 of FIG. 4, patient specific report 600-1 of FIG. 6A, patient specific report 600-2 of FIG. 6B, etc.) for one or more neuropsychiatric disorders at a client device (e.g., client device 300 of FIG. 1, client device 300 of FIG. 3, client device 300 of method 400 of FIG. 4, etc.).

The system 100 includes a display (e.g., display 208 of FIG. 2), one or more processors (e.g., CPU(s) 202 of FIG. 2), and first memory (e.g., memory 212 of FIG. 2) coupled to the one or more processors. The first memory 212 includes one or more programs configured to be executed by the one or more processors, such as reporting module 252. When executed by the processor, the one or more programs (e.g., reporting module 252) performs a method (e.g., method 400 of FIG. 4, method 500 of FIG. 5, etc.)

The client device includes a display (e.g., display 308 of FIG. 3), one or more processors (e.g., CPU(s) 302 of FIG. 3), and second memory (e.g., memory 312 of FIG. 3) coupled to the one or more processors. The second memory 312 includes one or more programs configured to be executed by the one or more processors, such as client application 320. When executed by the processor, the one or more programs (e.g., client application 320) performs a method (e.g., method 400 of FIG. 4, method 500 of FIG. 5, etc.)

In some embodiments, block 502 of the method 500 is conducted in accordance with one or more portions of block 402 of FIG. 4, as describe supra.

Block 504. Referring to block 504, in some embodiments, the method 500 includes determining a mental health pharmacogenetic profile for the subject. The mental health pharmacogenetic profile is determined by obtaining or having obtained a biological sample from the subject. The method 500 includes performing or having performed a genotyping assay on the biological sample to determine the allele status for a plurality of genomic loci for the subject. Each respective genomic loci in the plurality of genomic loci is associated with a therapeutic efficacy of a treatment, in a plurality of treatments, for a neuropsychiatric disorder.

In some embodiments, an allele detection system (e.g., allele detection system 200 of FIG. 1, allele detection system 200 of FIG. 2, allele detection system 200 of method 400 of FIG. 4, etc.) conducts the genotyping assay.

Block 506. Referring to block 506, the method 500 includes receiving, at the client device 300 a patient specific report (e.g., patient specific report 600 of method 400 of FIG. 4, patient specific report 600-1 of FIG. 6A, patient specific report 600-2 of FIG. 6B, etc.). The patient specific report includes the determination of the allele status for the plurality of genomic loci and one or more treatments for the neuropsychiatric disorder.

In some embodiments, block 506 of the method 500 is conducted in accordance with one or more portions of block 404 of FIG. 4, as describe supra.

Block 508. Referring to block 508, the method 500 includes displaying, on the display 308, in a graphical user interface, a respective graphical chart (e.g., graphical chart 602-1 of FIG. 6A) for each corresponding neuropsychiatric disorder in the one or more neuropsychiatric disorders. Each respective graphical chart 602 includes a respective first axis (e.g., first axis 604-1 of FIG. 6A), a respective second axis (e.g., second axis 604-2 of FIG. 6A), and one or more respective data plots (e.g., data plots 612 of FIG. 6A).

The respective first axis 604-1 is segmented into a respective plurality of bins (e.g., bins 606 of FIG. 6A). Each bin 606 in the respective plurality of bins 606 is associated with a respective independent first subset of genomic loci in the plurality of genomic loci. Each bin 606 in the respective plurality of bins 606 also represents a respective treatment class in a plurality of treatment classes.

The respective second axis 604-2 is associated with a parameter or a characteristic of an efficacy (e.g., third efficacy 608-4 of FIG. 6A) of a treatment for the corresponding neuropsychiatric disorder associated with the respective graphical chart 602.

Furthermore, each respective data plot 612 of the one or more respective data plots 612 represents a corresponding treatment, in the plurality of treatments, is within the corresponding bin 602 that is associated with the corresponding treatment, and is independently located relative to a position on the respective second axis 604-2 based on an allele status (e.g., allele status 242 of FIG. 2) determined for the subject (e.g., determined by genetic test result module 220 of FIG. 2), by the receiving, for the respective independent first subset of genomic loci associated with the corresponding bin 606.

In some embodiments, block 508 of the method 500 is conducted in accordance with one or more portions of block 406 of FIG. 4, as describe supra.

Block 510. Referring to block 510, in some embodiments, the method 500 includes, when a respective data plot 612 indicates that a first treatment, in the plurality of treatments, is better suited for treatment of the neuropsychiatric disorder in the individual than a second treatment, in the plurality of treatments, administering the first treatment to the subject. When the data plot 612 indicates that the second treatment is better suited for treatment of the neuropsychiatric disorder in the individual that the first treatment, administering the second treatment to the subject. In some embodiments, the indication that the first treatment is better suited for treatment of the neuropsychiatric disorder in the individual than the second treatment is based on the positions of the respective data plots 612 of the first treatment and the second treatment. For instance, referring to FIG. 6A, a third data plot 612-2 for a corresponding treatment of Exercise is positioned closer to a third efficacy 608-3, which is associated with a positive deviation in efficacy 608, than a first data plot 612-1 for a corresponding treatment of Olanzapine/Fluoxetine.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces described with respect to FIGS. 6A and 6B. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of displaying a patient specific report for one or more neuropsychiatric disorders at a client device, the client device comprising a display, one or more processors, and memory coupled to the one or more processors, the memory comprising one or more programs configured to be executed by the one or more processors, the method comprising:
receiving, from an allele detection system, the patient specific report comprising (i) a determination of an allele status for a plurality of genomic loci for a subject, wherein each respective genomic loci in the plurality of genomic loci is associated with (i) a therapeutic efficacy of a treatment, in a plurality of treatments, for at least one neuropsychiatric disorder in the one or more neuropsychiatric disorders, and (ii) one or more treatments of the at least one neuropsychiatric disorder; and
displaying, on the display, on a graphical user interface, a respective graphical chart for each corresponding neuropsychiatric disorder in the one or more neuropsychiatric disorders, wherein each respective graphical chart comprises:
a respective first axis that is segmented into a respective plurality of bins, wherein each bin in the respective plurality of bins (i) is associated with (i) a respective independent first subset of genomic loci in the plurality of genomic loci and (ii) represents a respective treatment class in a plurality of treatment classes,
a respective second axis associated with a parameter or a characteristic of an efficacy of a treatment for the corresponding neuropsychiatric disorder associated with the respective graphical chart, and
one or more respective data plots, wherein:
each respective data plot of the one or more respective data plots (i) represents a corresponding treatment, in the plurality of treatments, (ii) is within the corresponding bin that is associated with the corresponding treatment and (iii) is independently located relative to a position on the second axis based on an allele status determined for the subject, by the receiving, for the respective independent first subset of genomic loci associated with the corresponding bin, thereby visualizing one or more treatments of the one or more neuropsychiatric disorders.

2. The method of claim 1, wherein the respective graphical chart comprises a frame of reference associated with a first therapeutic efficacy of the second axis.

3. The method of claim 1, wherein each respective genomic loci in a respective first subset of genomic loci of a bin of the respective graphical chart is associated with at least one respective data plot of the one or more respective data plots.

4. The method of claim 2, wherein a distance from the frame of reference to the location of a respective data plot corresponds to a deviation in efficacy of the corresponding treatment relative to the first efficacy of the frame of reference.

5. The method of claim 2, wherein each respective data plot of the one or more respective data plots consumes a corresponding region of the graphical chart.

6. The method of claim 5, wherein the first therapeutic efficacy of the respective second axis is associated with a first hue and a relative distance from the frame of reference to each respective data plot shifts a color of the respective data plot from the first hue to a second hue as a function of the relative distance.

7. The method of claim 5, wherein each respective treatment in the one or more treatments is a corresponding pharmaceutical composition and wherein for a subset of data plots of the one or more respective data plots:
each respective region of a corresponding data plot in the subset of data plots comprises one or more graphical icons in a plurality of graphical icons, and
each respective graphical icon in the plurality of graphical icons represents a parameter or characteristic of the corresponding treatment represented by the corresponding data plot.

8. The method of claim 7, wherein the subset of data plots comprises each data plot of the one or more respective data plots that satisfies a threshold parameter or characteristic of a respective efficacy.

9. The method of claim 7, wherein the plurality of graphical icons comprises a first graphical icon representing a change in weight of the subject.

10. The method of claim 7, wherein the plurality of graphical icons comprises a second graphical icon representing a deviation in efficacy based on a characteristic of the subject.

11. The method of claim 7, wherein the plurality of graphical icons comprises:
a third graphical icon representing a positive deviation in efficacy of a pharmaceutical composition, and
a fourth graphical icon representing a negative deviation in efficacy of a pharmaceutical composition.

12. The method of claim 7, wherein the plurality of graphical icons comprises:
a fifth graphical icon representing a positive deviation in tolerance with consumption of a pharmaceutical composition, and
a sixth graphical icon representing a negative deviation in tolerance with consumption of a pharmaceutical composition.

13. The method of claim 7, wherein the plurality of graphical icons comprises a seventh graphical icon representing an adverse side effect with consumption of a pharmaceutical composition.

14. The method of claim 7, wherein the plurality of graphical icons comprises an eighth graphical icon representing a warning associated with consumption of a pharmaceutical composition.

15. The method of claim 7, wherein the plurality of graphical icons comprises:
a ninth graphical icon representing a positive deviation in exposure with consumption of a pharmaceutical composition, and
a tenth graphical icon representing a negative deviation in exposure with consumption of a pharmaceutical composition.

16. The method of claim 7, wherein the plurality of graphical icons comprises an eleventh graphical icon representing a negative deviation in exposure with consumption of a cytochrome P450 1A2 inhibitor pharmaceutical composition.

17. The method of claim 7, wherein the plurality of graphical icons comprises a twelfth graphical icon representing a prodrug pharmaceutical composition.

18. The method of claim 7, wherein each respective graphical icon in the plurality of graphical icons is provided a corresponding rank, and wherein each respective graphical icon of a respective region of a corresponding data plot is displayed in an ordered form by rank.

19. A system for displaying a patient specific report for one or more neuropsychiatric disorders comprising one or more processors, a display, and memory, wherein one or more programs is stored in the memory and configured to be executed by the one or more processors, the one or more programs including instructions for:
receiving, from an allele detection system, the patient specific report comprising (i) a determination of an allele status for a plurality of genomic loci for a subject, wherein each respective genomic loci in the plurality of genomic loci is associated with (i) a therapeutic efficacy of a treatment, in a plurality of treatments, for at least one neuropsychiatric disorder in the one or more neuropsychiatric disorders, and (ii) one or more treatments of the at least one neuropsychiatric disorder; and
displaying, on the display, on a graphical user interface, a respective graphical chart for each corresponding neuropsychiatric disorder in the one or more neuropsychiatric disorders, wherein each respective graphical chart comprises:
a respective first axis that is segmented into a respective plurality of bins, wherein each bin in the respective plurality of bins (i) is associated with (i) a respective independent first subset of genomic loci in the plurality of genomic loci and (ii) represents a respective treatment class in a plurality of treatment classes,
a respective second axis associated with a parameter or a characteristic of an efficacy of a treatment for the corresponding neuropsychiatric disorder associated with the respective graphical chart, and
one or more respective data plots, wherein:
each respective data plot of the one or more respective data plots (i) represents a corresponding treatment, in the plurality of treatments, (ii) is within the corresponding bin that is associated with the corresponding treatment and (iii) is independently located relative to a position on the second axis based on an allele status determined for the subject, by the receiving, for the respective independent first subset of genomic loci associated with the corresponding bin, thereby visualizing one or more treatments of the one or more neuropsychiatric disorders.

20. A non-transitory computer readable storage medium, wherein the non-transitory computer readable storage medium stores instructions, which when executed by a computer system, cause the computer system to perform a method comprising:
receiving, from an allele detection system, the patient specific report comprising (i) a determination of an allele status for a plurality of genomic loci for a subject, wherein each respective genomic loci in the plurality of genomic loci is associated with (i) a therapeutic efficacy of a treatment, in a plurality of treatments, for at least one neuropsychiatric disorder in the one or more neuropsychiatric disorders, and (ii) one or more treatments of the at least one neuropsychiatric disorder; and
displaying, on the display, on a graphical user interface, a respective graphical chart for each corresponding neuropsychiatric disorder in the one or more neuropsychiatric disorders, wherein each respective graphical chart comprises:
a respective first axis that is segmented into a respective plurality of bins, wherein each bin in the respective plurality of bins (i) is associated with (i) a respective independent first subset of genomic loci in the plurality of genomic loci and (ii) represents a respective treatment class in a plurality of treatment classes,
a respective second axis associated with a parameter or a characteristic of an efficacy of a treatment for the corresponding neuropsychiatric disorder associated with the respective graphical chart, and
one or more respective data plots, wherein:
each respective data plot of the one or more respective data plots (i) represents a corresponding treatment, in the plurality of treatments, (ii) is within the corresponding bin that is associated with the corresponding treatment and (iii) is independently located relative to a position on the second axis based on an allele status determined for the subject, by the receiving, for the respective independent first subset of genomic loci associated with the corresponding bin, thereby visualizing one or more treatments of the one or more neuropsychiatric disorders.
